(19) United States
(12) Patent Application Publication
(10) Pub. No.: US 2012/0269757 A1
(43) Pub. Date: Oct. 25, 2012

(54) PRODUCTS FOR KERATIN FIBERS, CONTAINING AT LEAST ONE SPECIAL AMPHIPHILIC CATIONIC POLYMER AND AT LEAST ONE SPECIAL CATIONIC POLYMER HAVING VINYLIMIDAZOLE STRUCTURAL UNITS

(76) Inventors: Burkhard Mueller, Hamburg (DE); Oliver Kuhnert, Duesseldorf (DE)

(21) Appl. No.: 13/537,133

(22) Filed: Jun. 29, 2012

Related U.S. Application Data
(63) Continuation of application No. PCT/EP2010/069150, filed on Aug. 12, 2010.

(30) Foreign Application Priority Data
Dec. 29, 2009 (DE) 10 2009 055 357.6

(51) Int. Cl. A61K 8/81; A61Q 5/00; A61Q 5/06

(52) U.S. Cl. 424/70.15

(57) ABSTRACT

Agents for treating keratin-containing fibers, particularly human hair, containing in a cosmetically acceptable carrier (a) at least one amphiphilic, cationic polymer having at least one structural unit of formula (I), at least one structural unit of formula (II) and at least one structural unit of formula (III),

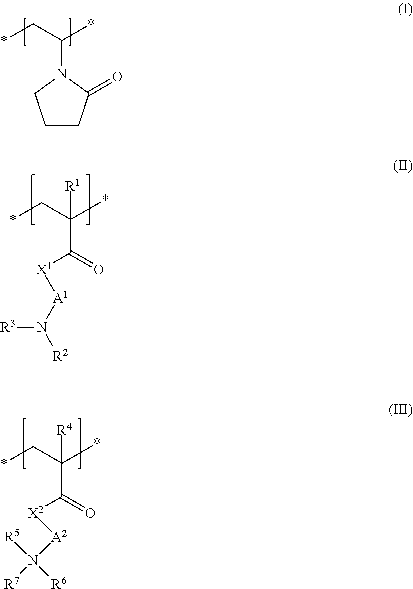

wherein R¹ and R⁴ are independently a hydrogen atom or a methyl group, X¹ and X² are independently an oxygen atom or an NH group, A¹ and A² are independently an ethane-1,2-diyl, propane-1,3-diyl or butane-1,4-diyl group, R², R³, R⁵ and R⁶ are independently a (C₁ to C₄) alkyl group, R⁷ is a (C₈ to C₃₀) alkyl group, and (b) at least one cationic polymer having at least one structural unit of formula (I), at least one structural unit of formula (V) and at least one structural unit of formula (VI)

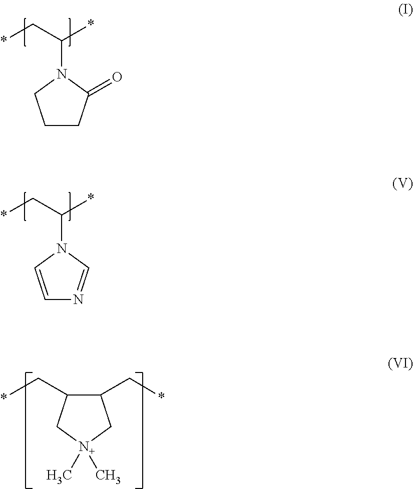

and use of those agents for temporarily shaping hair and for hair care.

### CROSS REFERENCE TO RELATED APPLICATIONS

The present application is a continuation of International Application No. PCT/EP2010/069150 filed 8 Dec. 2010, which claims priority to German Patent Application No. 10 2009 055 357.6, filed 29 Dec. 2009, both of which are incorporated herein by reference.

### BACKGROUND OF THE INVENTION

The present invention relates to agents for hair treatment, containing a combination of at least one special amphiphilic, cationic polymer and at least one special cationic polymer having vinylimidazole structural units, the use of said agents for the temporary shaping and/or for the care of keratin-containing fibers, and aerosol hair sprays/foams based on said agents.

Keratin-containing fibers include in principle all animal hair, for example, wool, horsehair, angora hair, fur, feathers and products or textiles manufactured therefrom. The keratinic fibers are, however, preferably human hair.

An attractive-looking hairstyle is today regarded as an essential element of a well-groomed exterior. The latest fashion trends mean that for many hair types, hairstyles that are regarded as fashionable can only be constructed or maintained for a lengthy period of up to several days using active fixing agents. Therefore hair treatment agents that provide a permanent or temporary shaping of the hair have an important role to play. Temporary shaping effects which offer good hold without adversely affecting the healthy appearance of the hair, such as its shine for example, can be achieved for example by means of hair sprays, hair waxes, hair gels, hair foams, blow-drying, etc.

Corresponding agents for temporary shaping conventionally contain synthetic polymers as the shaping component. Preparations containing a dissolved or dispersed polymer can be applied to the hair by means of propellants or via a pump mechanism. Hair gels and hair waxes in particular however are generally not applied directly to the hair but are distributed in the hair using a comb or the hands.

The most important property of an agent for temporarily shaping keratinic fibers, also referred to below as a styling agent, consists in giving the treated fibers in the created shape the strongest possible hold. If the keratinic fibers are human hair, this property is also described as a strong styling hold or a high degree of hold of the styling agent. The styling hold is substantially determined by the nature and amount of the synthetic polymer that is used, although the further constituents of the styling agent can also have an influence.

In addition to having a high degree of hold, styling agents also have to satisfy a whole series of further requirements. These can be broken down in approximate terms into properties of the hair, properties of the individual formulation, e.g. properties of the foam, gel or sprayed aerosol, and properties relating to the handling of the styling agent, particular importance being attached to the properties of the hair. Mention can be made in particular of moisture resistance, low tackiness and a balanced conditioning effect. As far as possible a styling agent should furthermore be universally suitable for all hair types and be gentle on the hair and skin.

In order to meet the diverse requirements, a large number of synthetic polymers for use in styling agents have already been developed. The polymers can be divided into cationic, anionic, non-ionic and amphoteric fixing polymers. The polymers ideally form a polymer film when applied to the hair, which on the one hand gives the hairstyle a strong hold but on the other is sufficiently flexible not to break under stress. If the polymer film is too brittle, film flakes or residues are formed which detach when the hair is moved and give the impression that the user of the corresponding styling agent has dandruff.

Developing styling agents that offer a combination of all desired properties continues to present difficulties. This is true in particular of the combination of strong hold on the one hand and an easy, uniform application onto the keratin-containing fibers on the other.

### SUMMARY OF THE INVENTION

The present invention therefore provides an agent for the temporary shaping and/or for the care of keratinic fibers that is distinguished by a high degree of hold or by a good caring effect, is gentle on the skin and in particular is extremely easy to handle during application on the keratin-containing fibers.

Surprisingly it has now been found that this can be achieved through a combination of special polymers. If formulated as hair foam, the compositions achieved with this combination even have particularly good foam parameters.

The present invention thus firstly provides an agent for treating keratin-containing fibers, particularly human hair, containing in a cosmetically acceptable carrier—
(a) at least one amphiphilic, cationic polymer, encompassing at least one structural unit of formula (I), at least one structural unit of formula (II) and at least one structural unit of formula (III),

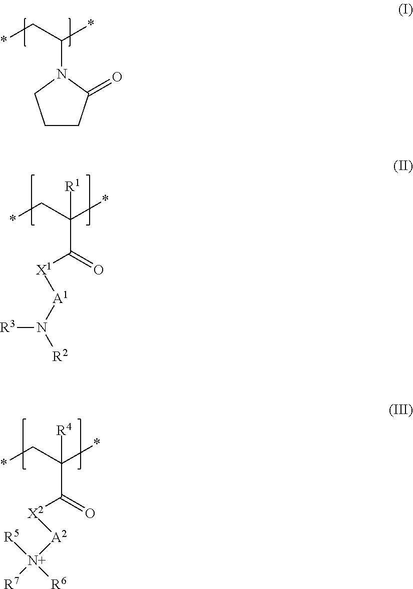

wherein

R¹ and R⁴ are independently a hydrogen atom or a methyl group,

X¹ and X² are independently an oxygen atom or an NH group,

A¹ and A² are independently an ethane-1,2-diyl, propane-1,3-diyl or butane-1,4-diyl group,

R², R³, R⁵ and R⁶ are independently a (C₁ to C₄) alkyl group, and

R⁷ is a (C₈ to C₃₀) alkyl group

and
(b) at least one cationic polymer comprising at least one structural unit of formula (I), at least one structural unit of formula (V) and at least one structural unit of formula (VI),

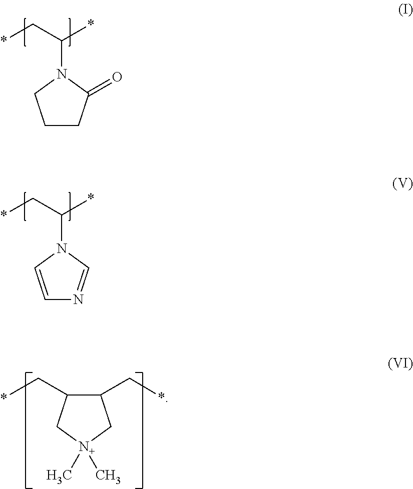

### DETAILED DESCRIPTION OF THE INVENTION

Fixing polymers contribute to maintaining and/or establishing volume and fullness in the hairstyle as a whole. These polymers are simultaneously also film-forming polymers and are therefore generally typical substances for hair treatment agents used for shaping, such as hair fixing agents, hair foams, hair waxes, hair sprays. Film formation may be entirely localized here and may bond only a few fibers together.

The curl retention test is frequently used as a test method for the fixing effect of a polymer.

Film-forming polymers are understood to be polymers which leave behind a continuous film on the skin, hair or nails when they dry. Such film formers can be used in a wide range of cosmetic products, such as for example face masks, make-up, hair fixing agents, hair sprays, hair gels, hair waxes, hair tonics, shampoos or nail varnishes. Such polymers are preferred in particular which have an adequate solubility in water or water/alcohol mixtures in order for them to be present in the agent according to the invention in completely dissolved form. The film-forming polymers can be of synthetic or natural origin.

Film-forming polymers are also understood according to the invention to be polymers which when used in a 0.01 to 20 wt. % aqueous, alcoholic or aqueous-alcoholic solution are capable of depositing a transparent polymer film on the hair.

In the above formulae and all subsequent formulae, a chemical bond marked with the symbol * denotes a free valence of the corresponding structural fragment.

The term “derivative” is understood according to the invention to mean derivatives of a chemical compound that can be prepared from this chemical compound by the structural change of a functional group of said chemical compound.

All possible physiologically tolerable anions, such as for example chloride, bromide, hydrogen sulfate, methyl sulfate, ethyl sulfate, tetrafluoroborate, phosphate, hydrogen phosphate, dihydrogen phosphate or p-toluenesulfonate, triflate, serve to offset the positive polymer charge in the agent according to the invention.

Examples of (C₁ to C₄) alkyl groups according to the invention are methyl, ethyl, propyl, isopropyl, butyl, sec-butyl, isobutyl, tert-butyl.

Examples of (C₈ to C₃₀) alkyl groups according to the invention are octyl (capryl), decyl (caprinyl), dodecyl (lauryl), tetradecyl (myristyl), hexadecyl (cetyl), octadecyl (stearyl), eicosyl (arachyl), docosyl (behenyl).

The amphiphilic, cationic polymers according to the invention preferably have a molecular weight M_{w} (weight average) from 10,000 g/mol to 50,000,000 g/mol, in particular from 50,000 g/mol to 5,000,000 g/mol, particularly preferably from 75,000 g/mol to 1,000,000 g/mol.

Preferred agents within the meaning of the invention contain the amphiphilic, cationic polymers (a) in an amount from 0.1 wt. % to 20.0 wt. % particularly preferably from 0.2 wt. % to 10.0 wt. %, most particularly preferably from 0.5 wt. % to 7.0 wt. %, relative in each case to the weight of the agent.

The properties of the agent according to the invention prove to be particularly advantageous if it is formulated as an aerosol spray, aerosol foam, pump spray or pump foam. This preferred form of formulation is described in detail further on.

According to the invention the following amphiphilic, cationic polymers (a) are preferably used in the agents according to the invention if the amphiphilic, cationic polymers (a) satisfy one or more of the following features in regard to the aforementioned formulae (I) to (III):
R¹ and R⁴ are each a methyl group,
X¹ is an NH group,
X² is an NH group,
A¹ and A² are independently ethane-1,2-diyl or propane-1,3-diyl,
R², R³, R⁵ and R⁶ are independently methyl or ethyl (preferably methyl).
R⁷ is a (C₁₀ to C₂₄) alkyl group, particularly decyl (caprinyl), dodecyl (lauryl), tetradecyl (myristyl), hexadecyl (cetyl), octadecyl (stearyl), eicosyl (arachyl) or docosyl (behenyl).

It is preferable according to the invention to select the structural unit of formula (II) from at least one structural unit of formula (II-1) to (II-8)—

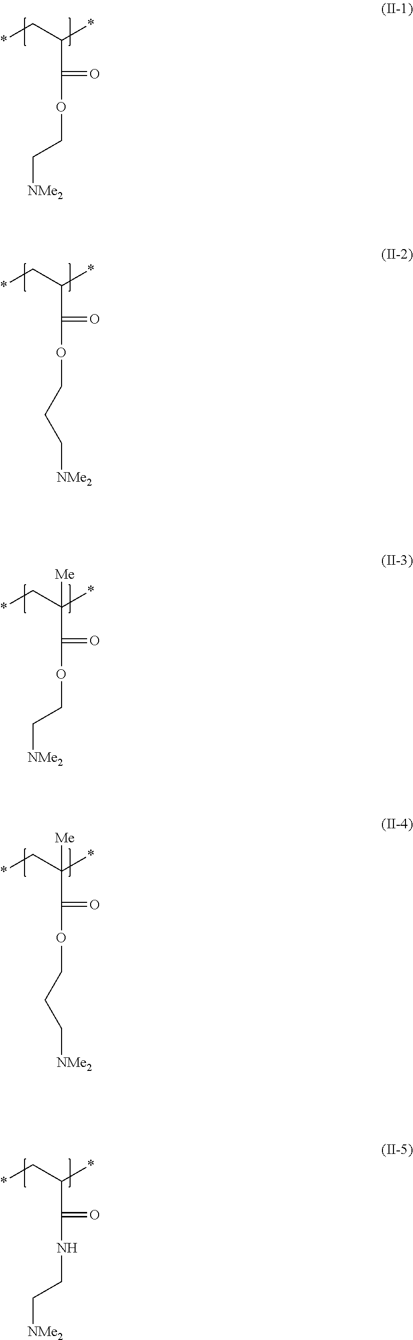

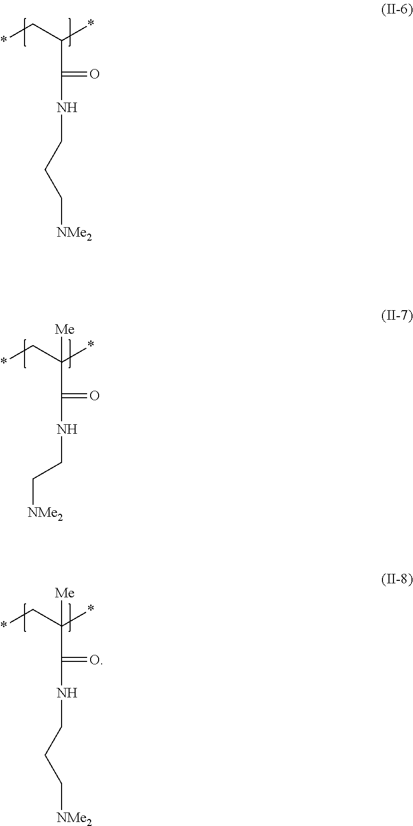

It has moreover proved particularly preferable to choose the structural unit according to formula (II-7) and/or formula (II-8) as the structural unit of formula (II). According to the invention the structural unit of formula (II-8) is a most particularly preferred structural unit.

With a view to achieving the object it has furthermore proved preferable for the structural unit of formula (III) to be selected from at least one structural unit of formulae (III-1) to (III-8)—

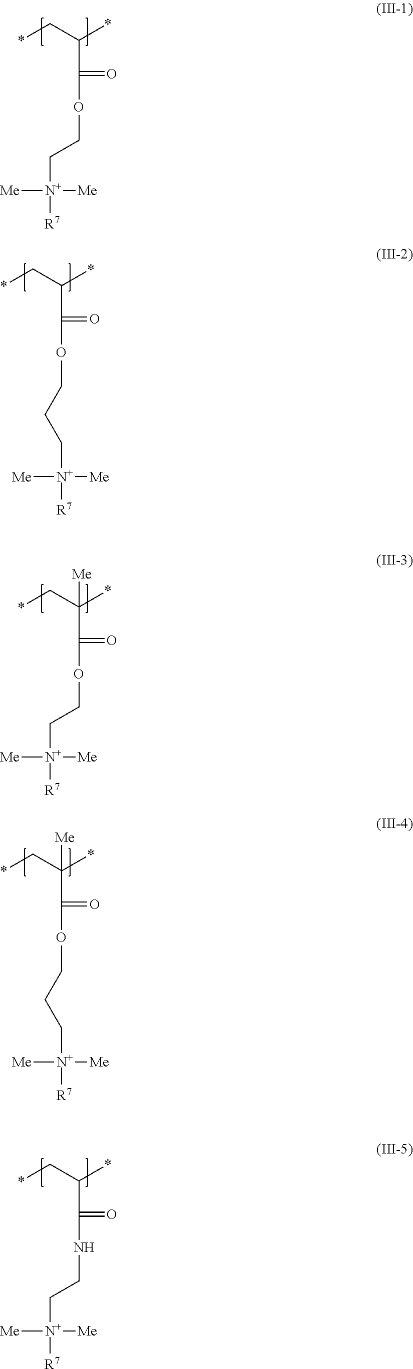

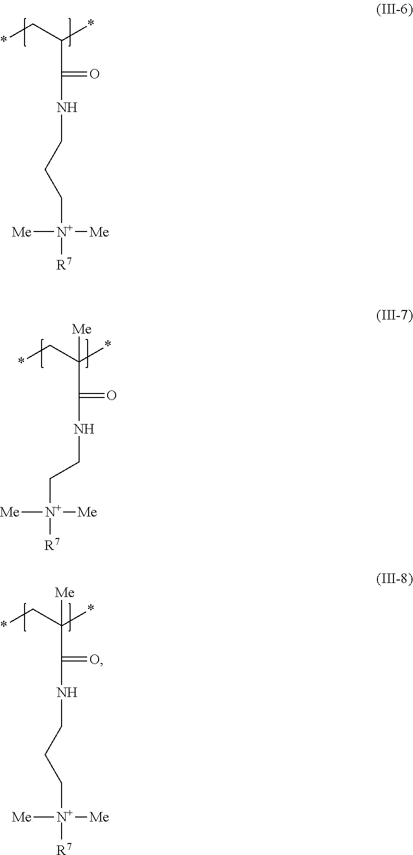

wherein R⁷ in each formula is a (C₈ to C₃₀) alkyl group.

The structural units of formulae (III-7) and/or (III-8), wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl), are regarded as a particularly preferred structural unit of formula (III). According to the invention, the structural unit of formula (III-8) is a most particularly preferred structural unit of formula (III).

An amphiphilic, cationic polymer that is most particularly preferably present in the agent according to the invention comprises at least one structural unit of formula (I), at least one structural unit of formula (II-8) and at least one structural unit of formula (III-8)—

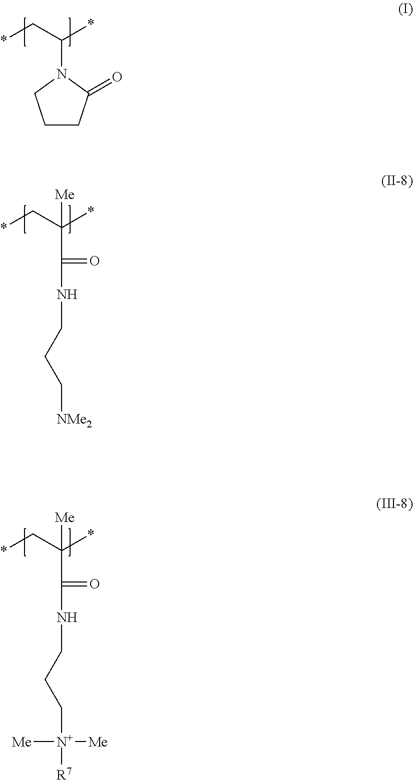

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl).

A most particularly preferred amphiphilic, cationic polymer according to the invention is the copolymer of N-vinylpyrrolidone, N-(3-dimethylaminopropyl)methacrylamide and 3-(methacryloylamino)propyl lauryl dimethylammonium chloride (INCI name: Polyquaternium-55), which is sold for example by ISP under the trade name Styleze® W10 or Styleze® W20 (10 wt. % active substance in W10, 20 wt. % active substance in W20, in water in each case, molecular weight 655,000).

In addition, agents according to the invention contain at least one cationic polymer (b), comprising at least one structural unit of formula (I), at least one structural unit of formula (V) and at least one structural unit of formula (VI)—

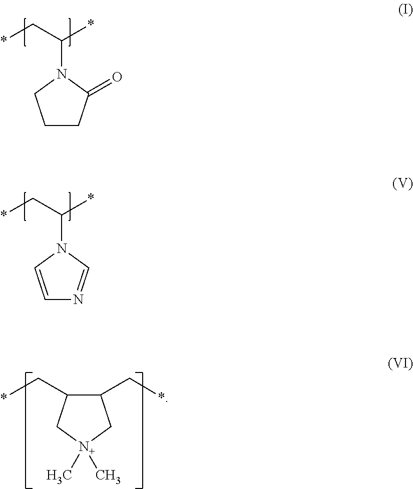

The cationic polymer (b) is preferably present in agents according to the invention in an amount from 0.01 wt. % to 20 wt. %, more preferably from 0.01 wt. % to 10.0 wt. %, even more preferably from 0.1 wt. % to 3 wt. %, based on total weight of the agent.

Preferably, polymer (b) has an average molecular weight M_{w} (weight average) from 50,000 to 250,000 g/mol, particularly from 75,000 to 200,000 g/mol.

In the cationic polymer, the structural units of formulae (I), (V) and (VI) can be randomly distributed or can be present in the form of polymeric or oligomeric blocks, such as blocks of poly[diallyldimethylammonium] units (particularly poly[diallyldimethylammonium chloride] units) and blocks of poly[N-vinylpyrrolidone/N-vinylimidazole] units. The blocks of poly[N-vinylpyrrolidone/N-vinylimidazole] units consist of structural units of the above formula (I) and the above formula (V), which are randomly distributed within the block.

Such a cationic polymer (b) thus includes at least one structural unit of the formula (Block-1) and at least one structural unit of the formula (Block-2)—

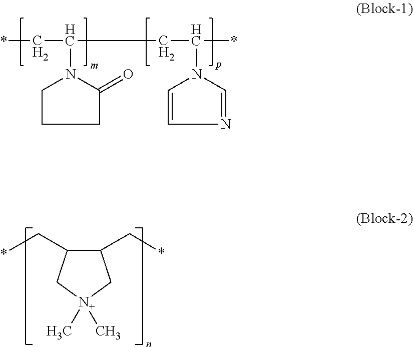

wherein
m and p are an integer number greater than 0,
the building blocks within the structural unit of formula (Block-1) are randomly distributed and n is an integer number greater than 1, particularly greater than 10.

A preferred cationic polymer (b) is a copolymer of N-vinylpyrrolidone, N-vinylimidazole and a diallydimethylammonium compound (in particular diallyldimethylammonium chloride). Such copolymers are known under the INCI name Polyquaternium-87 and are sold for example by BASF SE under the trade name Luviquat® Sensation in the form of an aqueous solution containing 27.5 wt. % of active substance. It is preferable according to the invention for the agents according to the invention to contain at least one polymer with the INCI name Polyquaternium-87.

A preferred embodiment is an agent for treating keratin-containing fibers, particularly human hair, containing in a cosmetically acceptable carrier—
(a) at least one amphiphilic, cationic polymer comprising at least one structural unit of formula (I), at least one structural unit of formula (II-8) and at least one structural unit of formula (III-8),

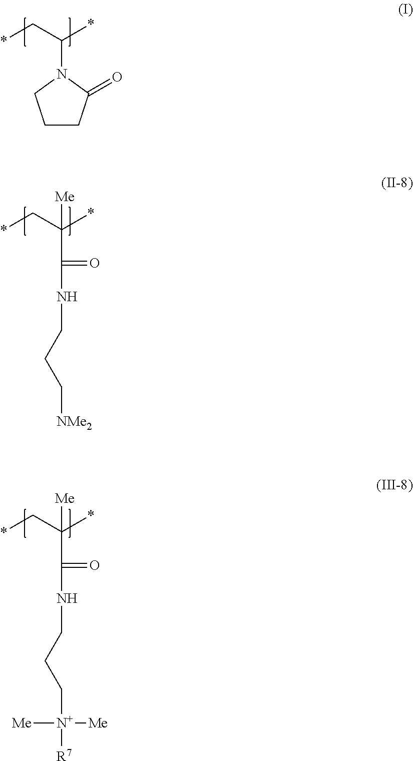

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl),
and
(b) at least one cationic copolymer of N-vinylpyrrolidone, N-vinylimidazole and a diallyldimethylammonium compound (particularly diallyldimethylammonium chloride).

A particularly preferred embodiment is an agent for treating keratin-containing fibers, particularly human hair, containing in a cosmetically acceptable carrier—
(a) Polyquaternium-55 as the amphiphilic, cationic polymer, and
(b) Polyquaternium-87.

The aforementioned preferred amounts are also preferred for this embodiment with necessary alterations.

A most particularly preferred embodiment of the invention is for the amphiphilic cationic polymer (a) to additionally include at least one structural unit of formula (IV)—

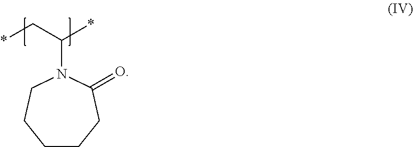

The aforementioned embodiments of formula (II) and (III) and the preferred amounts and molecular weights are also preferred for this embodiment with necessary alterations.

An amphiphilic, cationic polymer that is most particularly preferably contained in the agent according to the invention therefore includes at least one structural unit of formula (I), at least one structural unit of formula (II-8), at least one structural unit of formula (III-8) and at least one structural unit of formula (IV)—

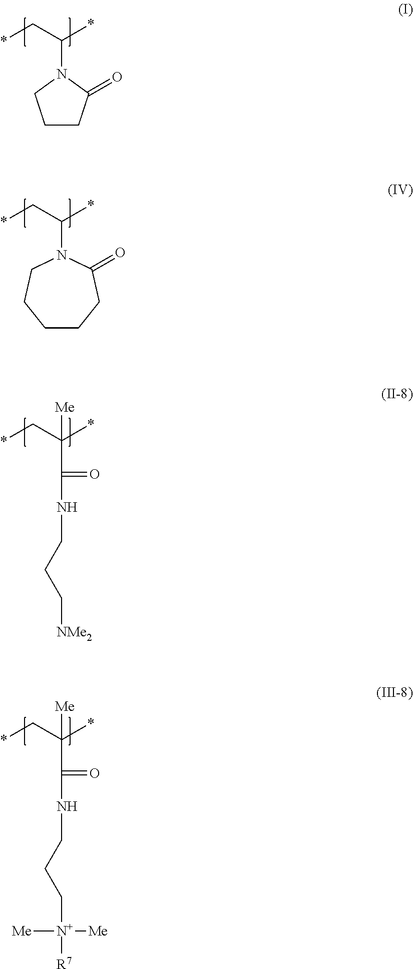

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl).

A most particularly preferred amphiphilic, cationic polymer according to the invention is the copolymer of N-vinylpyrrolidone, N-vinylcaprolactam, N-(3-dimethylaminopropyl)methacrylamide and 3-(methacryloylamino)propyl lauryl dimethylammonium chloride (INCI name: Polyquaternium-69), which is sold for example by ISP under the trade name AquaStyle® 300 (28-32 wt. % active substance in an ethanol-water blend, molecular weight 350,000).

A most particularly preferred embodiment is an agent for treating keratin-containing fibers, particularly human hair, containing in a cosmetically acceptable carrier—
(a) Polyquaternium-69 as the amphiphilic, cationic polymer, and
(b) Polyquaternium-87.

Agents according to the invention can additionally contain at least one further fixing polymer, selected from non-ionic polymers and/or from permanently cationic polymers and/or from amphoteric polymers, which differs from polymers (a) and (b).

In the context of a special embodiment the agent according to the invention can contain in addition to polymers (a) and (b) at least one film-forming non-ionic and/or fixing non-ionic polymer (c1).

Preferably, the fixing non-ionic polymers (c1) is chosen from at least one fixing non-ionic polymer containing at least one structural unit chosen from the structural units of formulae (I), (IV) and (M3)—

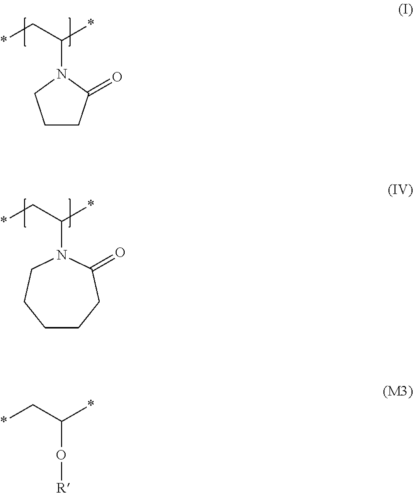

wherein R′ is a hydrogen atom or a (C₂ to C₁₈) acyl group.

According to the invention, non-ionic film-forming and/or non-ionic fixing polymers (c1) having at least one structural element of formula (M3) bearing a hydrogen atom, an acetyl group or a propanoyl group, particularly a hydrogen atom or an acetyl group, as R′, are preferred.

Non-ionic fixing polymers (c1) are preferably chosen from at least one polymer from:
homopolymers and non-ionic copolymers of N-vinylpyrrolidone,
polyvinyl alcohol, and
polyvinyl acetate.

Suitable polyvinylpyrrolidones include, for example, commercial products such as Luviskol® K 90 or Luviskol® K 85 from BASF SE.

Suitable polyvinyl alcohols are sold, for example, under the trade names Elvanol® by Du Pont or Vinol® 523/540 by Air Products.

Suitable polyvinyl acetate is sold, for example, as an emulsion under the trade name Vinac® by Air Products.

Agents containing as the fixing non-ionic polymer (c1) at least one polymer chosen from the group of—
polyvinylpyrrolidone, and
copolymers of N-vinylpyrrolidone and vinyl esters of carboxylic acids having 2 to 18 carbon atoms, particularly N-vinylpyrrolidone and vinyl acetate,
   are most particularly preferred according to the invention.

In the context of this embodiment, agents that are most particularly preferred are those containing in a cosmetically acceptable carrier—
(a) at least one amphiphilic, cationic polymer comprising at least one structural unit of formula (I), at least one structural unit of formula (II), at least one structural unit of formula (II-8) and at least one structural unit of formula (III-8),

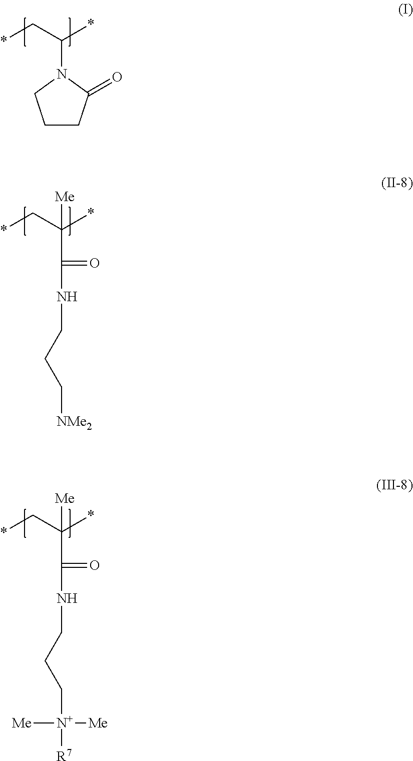

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl),
(b) at least one cationic polymer comprising at least one structural unit of formula (I), at least one structural unit of formula (V) and at least one structural unit of formula (VI),

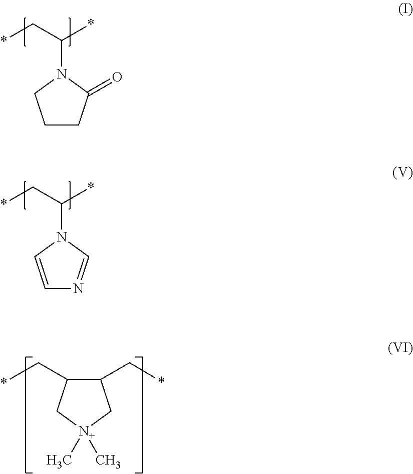

and
(c1) polyvinylpyrrolidone.

In the context of this embodiment, agents that are most particularly preferred are those containing in a cosmetically acceptable carrier—
(a) at least one amphiphilic, cationic polymer comprising at least one structural unit of formula (I), at least one structural unit of formula (II-8) and at least one structural unit of formula (III-8),

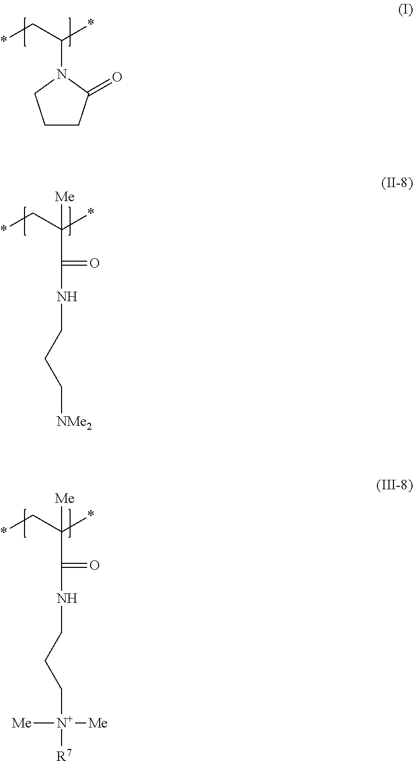

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl),
(b) at least one cationic polymer comprising at least one structural unit of formula (I), at least one structural unit of formula (V) and at least one structural unit of formula (VI),

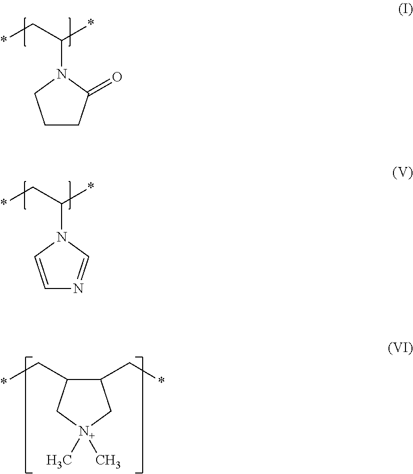

and
(c1) a copolymer produced from the monomers N-vinylpyrrolidone and vinyl acetate—particularly consisting of no further monomer.

Preferably, the molar ratio in the polymer of structural units contained from the N-vinylpyrrolidone monomer to structural units contained from the vinyl acetate monomer is in the range from 20 to 80 to 80 to 20, particularly from 30 to 70 to 70 to 30.

Suitable copolymers of vinylpyrrolidone and vinyl acetate are obtainable, for example, from BASF SE under the trademarks Luviskol® VA 37, Luviskol® VA 55, Luviskol® VA 64 and Luviskol® VA 73.

In the context of these embodiments the aforementioned preferred embodiments of the amphiphilic, cationic polymer (a) are preferred (see above).

All aforementioned preferred amounts in respect of the polymer components (a) and (b) of the agent according to the invention are also preferred for these embodiments with necessary alterations.

In the context of a special embodiment the agent according to the invention can contain in addition to polymers (a) and (b) at least one fixing, permanently cationic polymer (c2) that has at least one structural unit containing at least one permanently cationized nitrogen atom. The polymers (c2) are different from the amphiphilic, cationic polymers (a) and the polymers (b).

Permanently cationized nitrogen atoms are understood to be nitrogen atoms bearing a positive charge and thus forming a quaternary ammonium compound. Quaternary ammonium compounds are mostly produced by reacting tertiary amines with alkylating agents, such as for example methyl chloride, benzyl chloride, dimethyl sulfate, dodecyl bromide, but also ethylene oxide. Depending on the tertiary amine used, the following groups are known in particular: alkyl ammonium compounds, alkenyl ammonium compounds, imidazolinium compounds and pyridinium compounds.

Preferred agents within the meaning of the invention contain the film-forming cationic and/or fixing cationic polymers (c2) in an amount from 0.1 wt. % to 20.0 wt. % particularly preferably from 0.2 wt. % to 10.0 wt. %, most particularly preferably from 0.5 wt. % to 5.0 wt. %, relative in each case to the weight of the agent.

The cationic film-forming and/or cationic fixing polymers are particularly preferably selected according to the invention from cationic quaternized cellulose derivatives.

In the context of this embodiment, agents are particularly preferred which contain in a cosmetically acceptable carrier—
(a) at least one amphiphilic, cationic polymer comprising at least one structural unit of formula (I), at least one structural unit of formula (II) and at least one structural unit of formula (III),

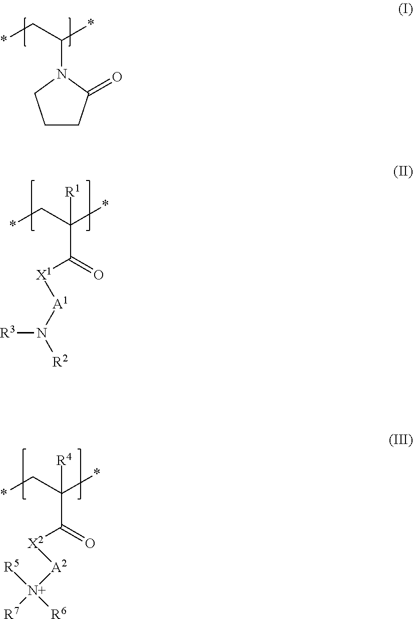

wherein
R¹ and R⁴ are independently a hydrogen atom or a methyl group,
X¹ and X² are independently an oxygen atom or an NH group,
A¹ and A² are independently an ethane-1,2-diyl, propane-1,3-diyl or butane-1,4-diyl group,
R², R³, R⁵ and R⁶ are independently a (C₁ to C₄) alkyl group, and
R⁷ is a (C₈ to C₃₀) alkyl group,
(b) at least one cationic polymer comprising at least one structural unit of formula (I), at least one structural unit of formula (V) and at least one structural unit of formula (VI),

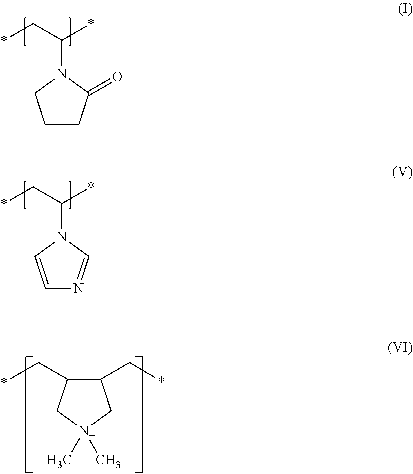

and
(c2) at least one additional cationic fixing polymer chosen from cationic, quaternized cellulose derivatives.

In the context of this embodiment, particularly preferred agents are in particular those which contain in a cosmetically acceptable carrier—
(a) at least one amphiphilic, cationic polymer comprising at least one structural unit of formula (I), at least one structural unit of formula (II-8) and at least one structural unit of formula (III-8),

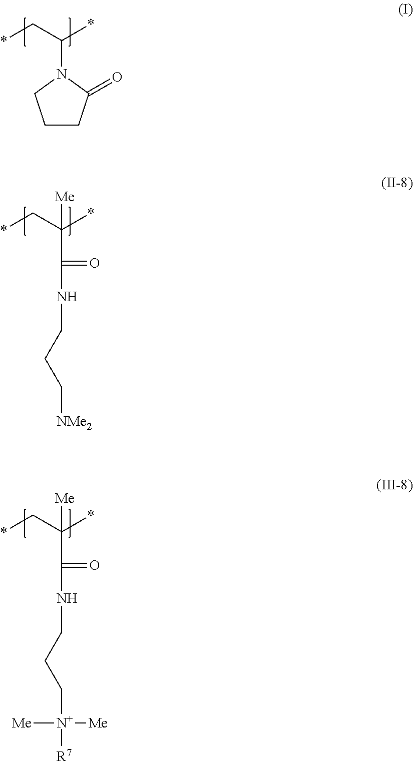

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl),
(b) at least one cationic polymer comprising at least one structural unit of formula (I), at least one structural unit of formula (V) and at least one structural unit of formula (VI),

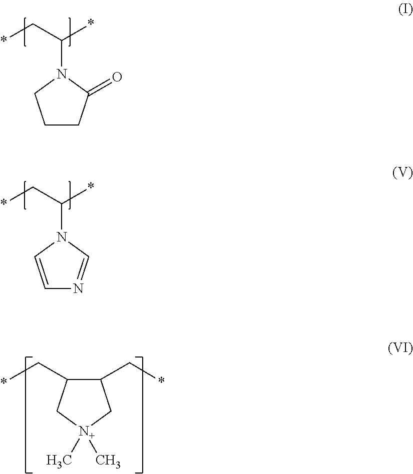

and
(c2) at least one additional cationic fixing polymer chosen from cationic, quaternized cellulose derivatives.

As a general rule, cationic, quaternized celluloses having more than one permanent cationic charge in a side chain are advantageous within the meaning of the invention.

Of these, cationic cellulose derivatives produced by reacting hydroxyethyl cellulose with a dimethyldiallylammonium reactant (particularly dimethyldiallylammonium chloride), optionally in the presence of further reactants, should be mentioned. Of these cationic celluloses, cationic celluloses with the INCI name Polyquaternium-4 are particularly suitable, and are sold, for example, by National Starch under the names Celquat® H 100, Celquat® L 200.

Thus, in the context of this embodiment, agents that are particularly preferred are those which contain in a cosmetically acceptable carrier—
(a) at least one amphiphilic, cationic polymer comprising at least one structural unit of formula (I), at least one structural unit of formula (II-8) and at least one structural unit of formula (III-8)

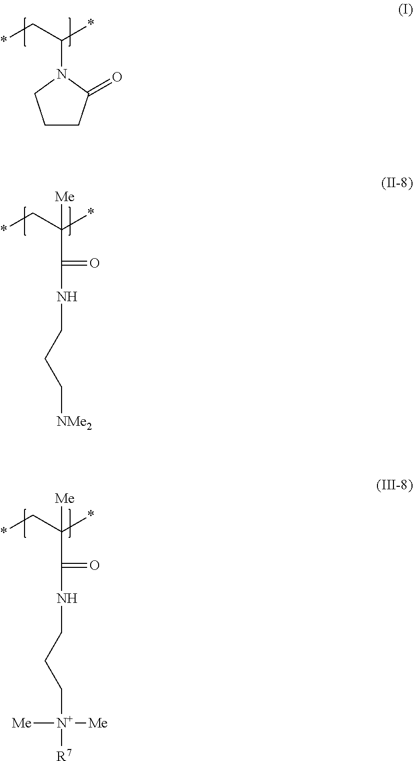

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl),
(b) at least one cationic polymer comprising at least one structural unit of formula (I), at least one structural unit of formula (V) and at least one structural unit of formula (VI),

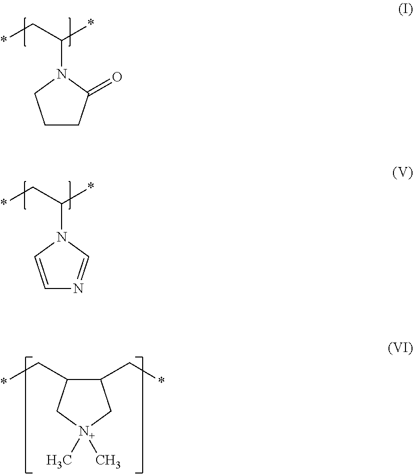

and
(c2) at least one additional fixing cationic polymer chosen from cationic, quaternized cellulose derivatives produced by reacting hydroxyethyl cellulose with a dimethyldiallylammonium reactant (particularly dimethyldiallylammonium chloride), optionally in the presence of further reactants.

In the context of these above embodiments the aforementioned preferred embodiments of the amphiphilic, cationic polymer (a) are suitable (see above). All aforementioned preferred amounts in respect of the polymer components (a) and (b) of the agent according to the invention are also suitable for these embodiments with necessary alterations.

Serving as the permanently cationic polymer (c2) that is likewise preferably suitable for use within the meaning of the invention are cationic film-forming and/or cationic fixing copolymers (c2) that contain at least one structural element of formula (M4)—

Within the meaning of the present invention agents are therefore preferred which contain in a cosmetically acceptable carrier—
(a) at least one amphiphilic, cationic polymer comprising at least one structural unit of formula (I), at least one structural unit of formula (II) and at least one structural unit of formula (III),

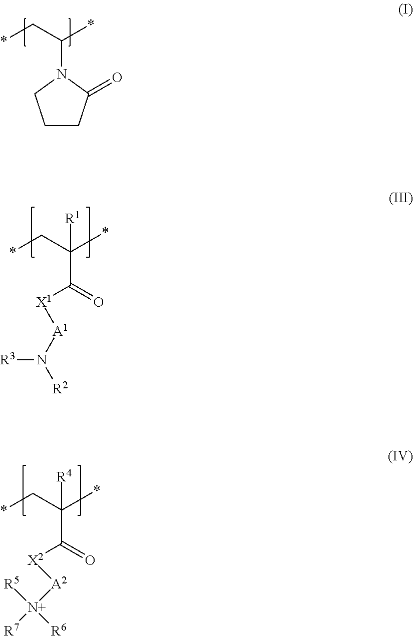

wherein
R¹ and R⁴ are independently a hydrogen atom or a methyl group,
X¹ and X² are independently an oxygen atom or an NH group,
A¹ and A² are independently an ethane-1,2-diyl, propane-1,3-diyl or butane-1,4-diyl group,
R², R³, R⁵ and R⁶ are independently a (C₁ to C₄) alkyl group, and
R⁷ is a (C₈ to C₃₀) alkyl group,
(b) at least one cationic polymer comprising at least one structural unit of formula (I), at least one structural unit of formula (V) and at least one structural unit of formula (VI),

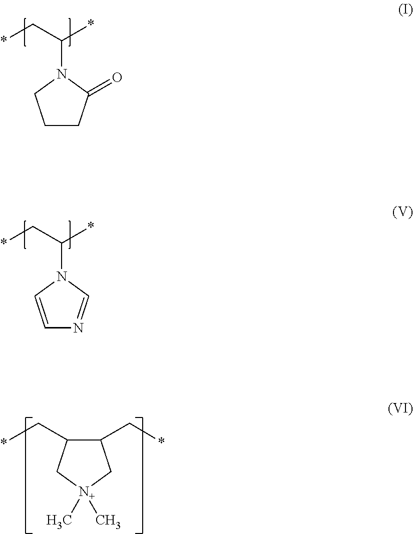

and
(c2) at least one additional cationic film-forming and/or cationic fixing polymer that as the at least one structural unit having a permanently cationized nitrogen atom contains at least one structural unit of formula (M4)

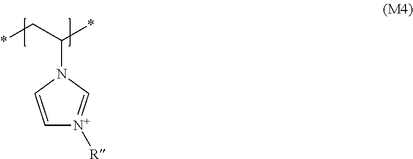

wherein R″ is a (C₁ to C₄) alkyl group, particularly a methyl group,
   and that additionally has at least one further cationic and/or non-ionic structural element.

Agents corresponding to this embodiment form transparent (i.e., non-turbid) compositions. Turbidity cannot be seen with the naked eye. Furthermore, agents of this embodiment additionally satisfy to an outstanding extent the advantageous parameters of strong degree of hold of the hairstyle, volume, or hair care.

All possible physiologically tolerable anions, such as chloride, bromide, hydrogen sulfate, methyl sulfate, ethyl sulfate, tetrafluoroborate, phosphate, hydrogen phosphate, dihydrogen phosphate or p-toluenesulfonate, triflate, serve to offset the positive polymer charge of component (c2).

In the context of this embodiment, preferred agents are in particular those which contain in a cosmetically acceptable carrier—
(a) at least one amphiphilic, cationic polymer comprising at least one structural unit of formula (I), at least one structural unit of formula (II-8) and at least one structural unit of formula (III-8)

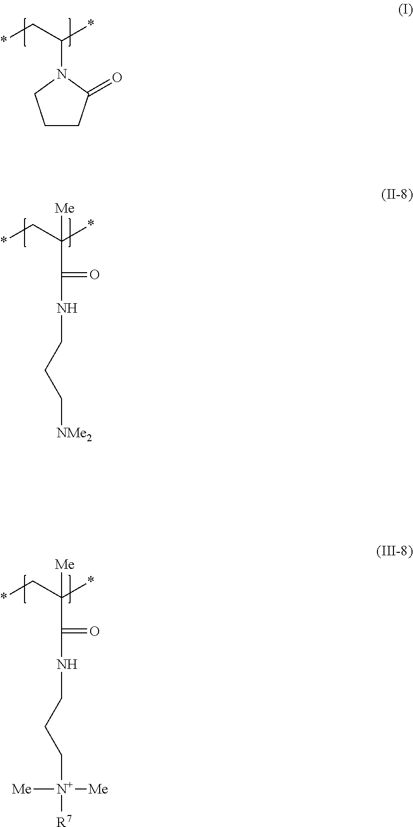

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl),
(b) at least one cationic polymer comprising at least one structural unit of formula (I), at least one structural unit of formula (V) and at least one structural unit of formula (VI),

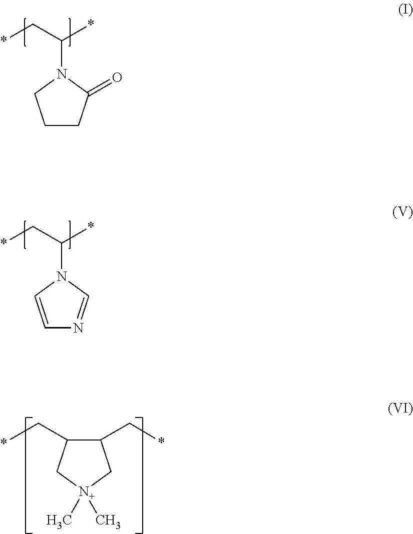

and
(c2) at least one cationic film-forming and/or cationic fixing polymer containing at least one structural element of formula (M4)

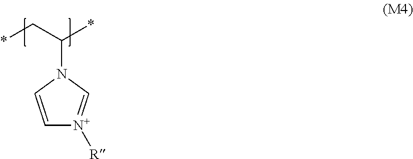

wherein R″ is a (C₁ to C₄) alkyl group, particularly a methyl group,
   and that additionally has at least one further cationic and/or non-ionic structural element.

In particular, the aforementioned polymer with the INCI name Polyquaternium-69 (see above) is particularly preferably suitable as the amphiphilic cationic polymer (a).

Regarding the offsetting of the positive polymer charge of component (c2), the details given above apply.

Preferably, the agent according to the invention contains, in addition to the amphiphilic cationic polymer (a), as cationic film-forming and/or cationic fixing polymer (c2) of this embodiment at least one copolymer (c2-1) that, in addition to at least one structural element of formula (M4), additionally has a structural element of formula (I)—

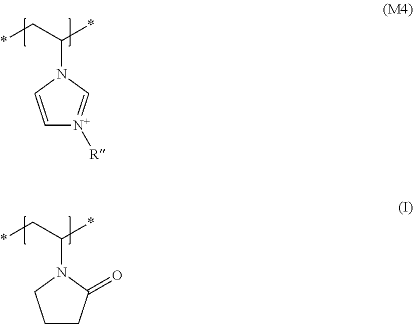

wherein R″ is a (C₁ to C₄) alkyl group, particularly a methyl group.

All possible physiologically tolerable anions, such as chloride, bromide, hydrogen sulfate, methyl sulfate, ethyl sulfate, tetrafluoroborate, phosphate, hydrogen phosphate, dihydrogen phosphate or p-toluene sulfonate, triflate, serve to offset the positive polymer charge of the copolymers (c2-1).

Most particularly preferred cationic film-forming and/or cationic fixing polymers as copolymers (c2-1) contain 10 to 30 mol %, preferably 15 to 25 mol % and in particular 20 mol % of structural units according to formula (M4) and 70 to 90 mol %, preferably 75 to 85 mol % and in particular 80 mol % of structural units according to formula (I).

It is particularly preferable here for the copolymers (c2-1) to contain, in addition to polymer units resulting from the incorporation of the cited structural units according to formula (M4) and (I) in the copolymer, a maximum of 5 wt. %, preferably a maximum of 1 wt. %, of polymer units resulting from the incorporation of other monomers. The copolymers (c2-1) are preferably made up exclusively of structural units of formula (M4) with R″=methyl and (I) and can be described by the general formula (Polyl)—

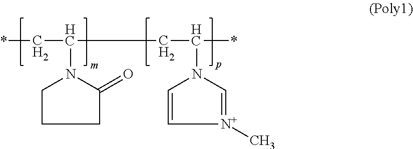

wherein m and p vary according to the molar mass of the polymer and are not intended to suggest that these are block copolymers. Structural units of formula (M4) and formula (I) can rather be randomly distributed in the molecule.

If a chloride ion is used to offset the positive charge of the polymer of formula (Polyl), these N-methyl vinylimidazole/vinylpyrrolidone copolymers are referred to under INCI nomenclature as Polyquaternium-16 and are available, for example, from BASF under the trade names Luviquat® Style, Luviquat® FC 370, Luviquat® FC 550, Luviquat® FC 905 and Luviquat® HM 552.

If a methosulfate is used to offset the positive charge of the polymer of formula (Polyl), these N-methyl vinylimidazole/vinylpyrrolidone copolymers are referred to under INCI nomenclature as Polyquaternium-44 and are available, for example, from BASF under the trade name Luviquat® UltraCare.

Particularly preferred agents according to the invention contain a copolymer (c2-1), in particular of the formula (Polyl), having molar masses within a defined range. Preferred agents are those in which copolymer (c2-1) has a molar mass of 50 to 400 kDa, preferably 100 to 300 kDa, more preferably 150 to 250 kDa and in particular 190 to 210 kDa.

Agents according to the invention can also contain, in addition to copolymer(s) (c2-1) or in its or their place, copolymers (c2-2) which, as additional structural units starting from copolymer (c2-1), have structural units of formula (IV)—

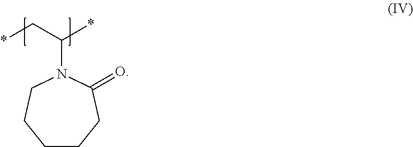

Further particularly preferred agents according to the invention thus contain as cationic film-forming and/or cationic fixing polymer (c2) at least one copolymer (c2-2) containing at least one structural unit according to formula (M4-a) and at least one structural unit according to formula (I) and at least one structural unit according to formula (IV)—

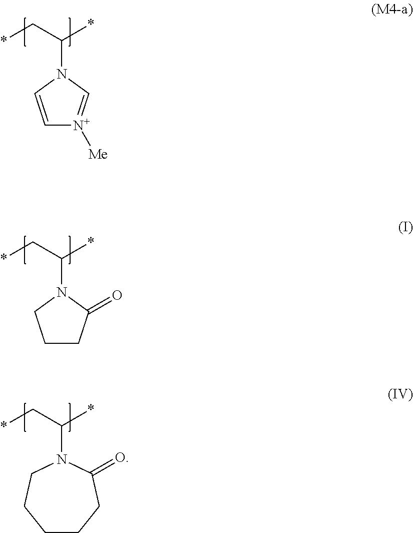

Here too it is particularly preferable for the copolymers (c2-2) to contain, in addition to polymer units resulting from the incorporation of the cited structural units according to formula (M4-a), (I) and (IV) in the copolymer, a maximum of 5 wt. %, preferably a maximum of 1 wt. %, of polymer units resulting from the incorporation of other monomers. The copolymers (c2-2) are preferably made up exclusively of structural units of formulae (M4-a), (I) and (IV) and can be described by the general formula (Poly2)—

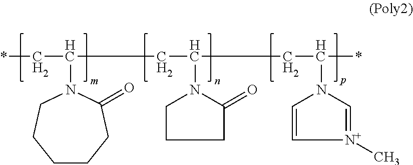

wherein m, n and p vary according to the molar mass of the polymer and are not intended to suggest that these are block copolymers. Structural units of this formula can instead be randomly distributed in the molecule.

All possible physiologically tolerable anions, such as chloride, bromide, hydrogen sulfate, methyl sulfate, ethyl sulfate, tetrafluoroborate, phosphate, hydrogen phosphate, dihydrogen phosphate or p-toluene sulfonate, triflate, serve to offset the positive polymer charge of component (c2-2).

If a methosulfate is used to offset the positive charge of the polymer of formula (Poly2), such N-methyl vinylimidazole/vinylpyrrolidone/vinylcaprolactam copolymers are referred to under INCI nomenclature as Polyquaternium-46 and are available for example from BASF under the trade name Luviquat® Hold.

Most particularly preferred copolymers (c2-2) contain 1 to 20 mol. %, preferably 5 to 15 mol. % and in particular 10 mol % of structural units according to formula (M4-a) and 30 to 50 mol %, preferably 35 to 45 mol % and in particular 40 mol % of structural units according to formula (I) and 40 to 60 mol %, preferably 45 to 55 mol % and in particular 60 mol % of structural units according to formula (IV).

Particularly preferred agents according to the invention contain a copolymer (c2-2) having molar masses within a defined range. Agents according to the invention are preferred here in which the copolymer (c2-2) has a molar mass of 100 to 1000 kDa, preferably 250 to 900 kDa, more preferably 500 to 850 kDa and in particular 650 to 710 kDa.

As film-forming cationic and/or fixing cationic polymer (c2) the agents according to the invention can also contain, in addition to the copolymer(s) (c2-1) and/or (c2-2) or in its or their place, copolymers (c2-3) which as structural units have structural units of formulae (M4-a) and (I) as well as further structural units from the group of vinylimidazole units and further structural units from the group of acrylamide and/or methacrylamide units.

Further particularly preferred agents according to the invention contain as cationic film-forming and/or cationic fixing polymer (c2) at least one copolymer (c2-3) containing at least one structural unit according to formula (M4-a) and at least one further structural unit according to formula (I) and at least one structural unit according to formula (V) and at least one structural unit according to formula (IX)—

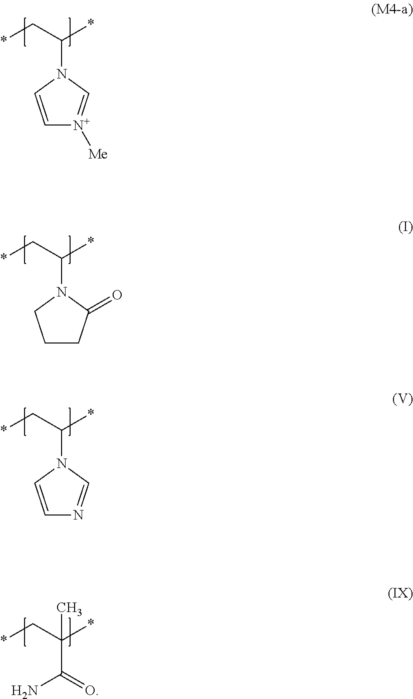

Here too it is particularly preferable for copolymers (c2-3) to contain, in addition to polymer units resulting from the incorporation of the cited structural units according to formula (M4-a), (I), (V) and (IX) in the copolymer, a maximum of 5 wt. %, preferably a maximum of 1 wt. %, of polymer units resulting from the incorporation of other monomers. Copolymers (c2-3) are preferably made up exclusively of structural units of formulae (M4-a), (I), (V) and (IX) and can be described by the general formula (Poly3)—

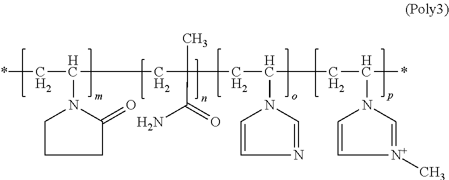

wherein m, n, o and p vary according to the molar mass of the polymer and are not intended to suggest that these are block copolymers. Structural units of formulae (M4-a), (I), (V) and (IX) can instead be randomly distributed in the molecule.

All possible physiologically tolerable anions, such as chloride, bromide, hydrogen sulfate, methyl sulfate, ethyl sulfate, tetrafluoroborate, phosphate, hydrogen phosphate, dihydrogen phosphate or p-toluene sulfonate, triflate, serve to offset the positive polymer charge of component (c2-3).

If a methosulfate is used to offset the positive charge of the polymer of formula (Poly3), such N-methyl vinylimidazole/vinylpyrrolidone/vinylimidazole/methacrylamide copolymers are referred to under INCI nomenclature as Polyquaternium-68 and are available, for example, from BASF under the trade name Luviquat® Supreme.

Most particularly preferred copolymers (c2-3) contain 1 to 12 mol. %, preferably 3 to 9 mol. % and in particular 6 mol % of structural units according to formula (M4-a), and 45 to 65 mol %, preferably 50 to 60 mol % and in particular 55 mol % of structural units according to formula (I) and 1 to 20 mol %, preferably 5 to 15 mol % and in particular 10 mol % of structural units according to formula (V), and 20 to 40 mol %, preferably 25 to 35 mol % and in particular 29 mol % of structural units according to formula (IX).

Particularly preferred agents according to the invention contain a copolymer (c2-3) having molar masses within a defined range. Agents according to the invention are preferred here in which the copolymer (c2-3) has a molar mass of 100 to 500 kDa, preferably 150 to 400 kDa, more preferably 250 to 350 kDa and in particular 290 to 310 kDa.

Of the additional film-forming cationic and/or fixing polymers chosen from cationic polymers (c2) having at least one structural element of formula (M4) above, the following are preferred:
vinylpyrrolidone/1-vinyl-3-methyl-1H-imidazolium chloride copolymers (e.g., those obtainable with the INCI name Polyquaternium-16 under the trade names Luviquat® Style, Luviquat® FC 370, Luviquat® FC 550, Luviquat® FC 905 and Luviquat® HM 552 (BASF SE)),
vinylpyrrolidone/1-vinyl-3-methyl-1H-imidazolium methyl sulfate copolymers (e.g., those obtainable with the INCI name Polyquaternium-44 under the trade names Luviquat® Care (BASF SE)),
vinylpyrrolidone/vinylcaprolactam/1-vinyl-3-methyl-1H-imidazolium terpolymer (e.g., those obtainable with the INCI name Polyquaternium-46 under the trade names Luviquat® Care or Luviquat® Hold (BASF SE)),
vinylpyrrolidone/methacrylamide/vinylimidazole/1-vinyl-3-methyl-1H-imidazolium methyl sulfate copolymer (e.g., those obtainable with the INCI name Polyquatemium-68 under the trade name Luviquat® Supreme (BASF SE)),
   and mixtures of these polymers.

In the context of this embodiment the aforementioned preferred embodiments of the amphiphilic, cationic polymer (a) are preferred (see above). All aforementioned preferred amounts in respect of the polymer components (a) and (b) of the agent according to the invention are also preferred for these embodiments with necessary alterations.

To intensify the effect according to the invention the agents according to the invention preferably additionally contain at least one surfactant, with non-ionic, anionic, cationic and ampholytic surfactants being suitable in principle. The group of ampholytic or amphoteric surfactants encompasses zwitterionic surfactants and ampholytes. According to the invention the surfactants can already have an emulsifying action.

The additional surfactants are preferably contained in the agent according to the invention in an amount from 0.01 wt. % to 5 wt. %, particularly preferably from 0.05 wt. % to 0.5 wt. %, relative in each case to the weight of the agent.

It has proved particularly preferable for the agents according to the invention additionally to contain at least one non-ionic surfactant.

Non-ionic surfactants contain as a hydrophilic group a polyol group, a polyalkylene glycol ether group or a combination of a polyol and polyglycol ether group, for example.

Such compounds include:
addition products of 2 to 100 mol of ethylene oxide and/or 1 to 5 mol of propylene oxide with linear and branched fatty alcohols having 8 to 30 C atoms, with fatty acids having 8 to 30 C atoms and with alkyl phenols having 8 to 15 C atoms in the alkyl group,
addition products of 2 to 50 mol of ethylene oxide and/or 1 to 5 mol of propylene oxide with linear and branched fatty alcohols having 8 to 30 C atoms, with fatty acids having 8 to 30 C atoms and with alkylphenols having 8 to 15 C atoms in the alkyl group, end-capped with a methyl or C₂ to C₆ alkyl residue, such as for example the types available under the commercial names Dehydrol® LS, Dehydrol® LT (Cognis),
C₁₂-C₃₀ fatty acid monoesters and diesters of addition products of 1 to 30 mol of ethylene oxide with glycerol,
addition products of 5 to 60 mol of ethylene oxide with castor oil and hydrogenated castor oil,
polyol fatty acid esters, such as the commercial product Hydagen® HSP (Cognis) or Sovermol types (Cognis),
alkoxylated triglycerides,
alkoxylated fatty acid alkyl esters of formula (E4-I)

R¹CO—(OCH₂CHR²)_{w}OR³ (E4-I)
wherein R¹CO is a linear or branched, saturated and/or unsaturated acyl residue having 6 to 22 carbon atoms, R² is hydrogen or methyl, R³ is linear or branched alkyl residues having 1 to 4 carbon atoms, and w is a number from 1 to 20,
amine oxides,
hydroxy mixed ethers, such as are described in DE-OS 19738866,
sorbitan fatty acid esters and addition products of ethylene oxide with sorbitan fatty acid esters such as for example polysorbates,
sugar fatty acid esters and addition products of ethylene oxide with sugar fatty acid esters,
addition products of ethylene oxide with fatty acid alkanol amides and fatty amines,
sugar surfactants of the alkyl and alkenyl oligoglycoside type according to formula (E4-II),

R⁴O-[G]ₚ (E4-II)
wherein R⁴ is an alkyl or alkenyl residue having 4 to 22 carbon atoms, G is a sugar residue having 5 or 6 carbon atoms, and p is a number from 1 to 10. They can be obtained by relevant methods of preparative organic chemistry.

Alkylene oxide addition products with saturated linear fatty alcohols and fatty acids each containing 2 to 100 mol of ethylene oxide per mol of fatty alcohol or fatty acid are most particularly preferred non-ionic surfactants. Preparations having outstanding properties are likewise obtained if they contain C₁₂-C₃₀ fatty acid mono- and diesters of addition products of 1 to 30 mol of ethylene oxide with glycerol and/or addition products of 5 to 60 mol of ethylene oxide with castor oil and hydrogenated castor oil as non-ionic surfactants.

Both products having a “normal” homolog distribution and those having a narrow homolog distribution can be used for the surfactants that are addition products of ethylene and/or propylene oxide with fatty alcohols or derivatives of these addition products. “Normal” homolog distribution refers to mixtures of homologs which are obtained by reacting fatty alcohol and alkylene oxide using alkali metals, alkali hydroxides or alkali alcoholates as catalysts. Narrow homolog distributions on the other hand are obtained when for example hydrotalcites, alkaline-earth metal salts of ether carboxylic acids, alkaline-earth metal oxides, hydroxides or alcoholates are used as catalysts. The use of products having a narrow homolog distribution can be preferred.

Agents according to the invention most particularly preferably contain at least one addition product of 15 to 100 mol of ethylene oxide, in particular of 15 to 50 mol of ethylene oxide, with a linear or branched (in particular linear) fatty alcohol having 8 to 22 carbon atoms as the surfactant. This is most particularly preferably Ceteareth-15, Ceteareth-25 or Ceteareth-50, which are sold respectively as Eumulgin® CS 15 (COGNIS), Cremophor A25 (BASF SE) and Eumulgin® CS 50 (COGNIS).

All anionic surface-active substances suitable for use on the human body are suitable in principle as anionic surfactants. These have the characterizing feature of a water-solubilizing anionic group such as for example a carboxylate, sulfate, sulfonate or phosphate group and a lipophilic alkyl group having approximately 8 to 30 C atoms. The molecule can additionally contain glycol or polyglycol ether groups, ester, ether and amide groups and hydroxyl groups. Examples of suitable anionic surfactants, each in the form of the sodium, potassium and ammonium salts as well as the mono-, di- and trialkanolammonium salts having 2 to 4 C atoms in the alkanol group, are—
linear and branched fatty acids having 8 to 30 C atoms (soaps),
ether carboxylic acids of the formula R—O—(CH₂—CH₂O)ₓ—CH₂—COOH, in which R is a linear alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
alkyl sulfates and alkyl polyglycol ether sulfates of the formula R—O(CH₂—CH₂O)ₓ—OSO₃H, in which R is a preferably linear alkyl group having 8 to 30 C atoms and x=0 or 1 to 12,
mixtures of surface-active hydroxyl sulfonates,
sulfated hydroxyalkyl polyethylene and/or hydroxyalkylene propylene glycol ethers,
sulfonates of unsaturated fatty acids having 8 to 24 C atoms and 1 to 6 double bonds,
esters of tartaric acid and citric acid with alcohols that are addition products of around 2 to 15 molecules of ethylene oxide and/or propylene oxide with fatty alcohols having 8 to 22 C atoms,
alkyl and/or alkenyl ether phosphates of the formula (E1-I),

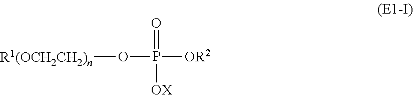

wherein R¹ is preferably an aliphatic hydrocarbon residue having 8 to 30 carbon atoms, R² is hydrogen, a (CH₂CH₂O)ₙR¹ residue or X, n is a number from 1 to 10, and X is hydrogen, an alkali or alkaline-earth metal or NR³R⁴R⁵R⁶, with R³ to R⁶ independently being hydrogen or a C₁ to C₄ hydrocarbon residue,
sulfated fatty acid alkylene glycol esters of the formula (E1-II)

R⁷CO(AlkO)ₙSO₃M (E1-II)
wherein R⁷CO is a linear or branched, aliphatic, saturated and/or unsaturated acyl residue having 6 to 22 C atoms, Alk is CH₂CH₂, CHCH₃CH₂ and/or CH₂CHCH₃, n is a number from 0.5 to 5, and M is a cation such as are described in DE-OS 197 36 906,
monoglyceride sulfates and monoglyceride ether sulfates of the formula (E1-III)

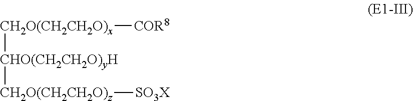

wherein R⁸CO is a linear or branched acyl residue having 6 to 22 carbon atoms, x, y and z in total is 0 or a number from 1 to 30, preferably 2 to 10, and X is an alkali or alkaline-earth metal. Typical examples of suitable monoglyceride (ether) sulfates within the meaning of the invention are the reaction products of lauric acid monoglyceride, coconut fatty acid monoglyceride, palmitic acid monoglyceride, stearic acid monoglyceride, oleic acid monoglyceride and tallow fatty acid monoglyceride as well as the ethylene oxide adducts thereof with sulfur trioxide or chlorosulfonic acid in the form of their sodium salts. Monoglyceride sulfates of the formula (E1-III) are preferably used wherein R⁸CO is a linear acyl residue having 8 to 18 carbon atoms,
amide ether carboxylic acids, and
condensation products of C₈ to C₃₀ fatty alcohols with protein hydrolysates and/or amino acids and derivatives thereof, known to one skilled in the art as protein fatty acid condensates, such as the Lamepon® types, Gluadin® types, Hostapon® KCG or Amisoft® types.

Preferred anionic surfactants are alkyl sulfates, alkyl polyglycol ether sulfates and ether carboxylic acids having 10 to 18 C atoms in the alkyl group and up to 12 glycol ether groups in the molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 oxyethyl groups, monoglyceride sulfates, alkyl and alkenyl ether phosphates and protein fatty acid condensates.

Particularly preferably suitable for use according to the invention are cationic surfactants of the quaternary ammonium compound, esterquat and amidoamine type. Preferred quaternary ammonium compounds are ammonium halides, particularly chlorides and bromides, such as alkyl trimethylammonium chlorides, dialkyl dimethylammonium chlorides and trialkyl methylammonium chlorides. The long alkyl chains of these surfactants preferably have 10 to 18 carbon atoms, such as for example in cetyl trimethylammonium chloride, stearyl trimethylammonium chloride, distearyl dimethylammonium chloride, lauryl dimethylammonium chloride, lauryl dimethyl benzylammonium chloride and tricetyl methylammonium chloride. Further preferred cationic surfactants are the imidazolium compounds known under the INCI names Quaternium-27, Quaternium-87 and Quaternium-91.

It is most particularly preferable according to the invention for the agents according to the invention to contain as the cationic surfactant at least one di(oligooxyethyl)ammonium salt or tris(oligooxyethyl)ammonium salt as the cationic surfactant. It is preferable for the agent according to the invention to contain at least one cationic surfactant of formula (X)—

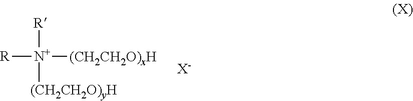

wherein
x and y are independently an integer number greater than 0,
R is a (C₈ to C₂₀) alkyl group or a (C₈ to C₂₀) alkenyl group,
R′ is a *—(CH₂CH₂O)_{z}H group wherein z is an integer greater than 0, a (C₈ to C₂₀) alkyl group or a (C₈ to C₂₀) alkenyl group, and
X⁻ is a physiologically tolerable anion.

Examples of (C₈ to C₃₀) alkyl groups according to the invention are octyl (capryl), decyl (caprinyl), dodecyl (lauryl), tetradecyl (myristyl), hexadecyl (cetyl), octadecyl (stearyl), eicosyl (arachyl), docosyl (behenyl). Examples of (C₈ to C₂₀) alkenyl groups according to the invention are oleyl, linolyl, linolenyl.

Preferably, the cationic surfactant of formula (X) has a molecular weight from 550 to 2500 g/mol, particularly preferably from 600 to 1000 g/mol.

R according to formula (X) is preferably dodecyl, tetradecyl, hexadecyl or octadecyl.

Preferably, R′ according to formula (X) is a *—(CH₂CH₂O)_{z}H group, wherein z is an integer number greater than 0.

X⁻ according to formula (X) is preferably chloride, phosphate or dihydrogen phosphate.

It is furthermore preferable if according to formula (X) x and y (and also z if R′ is the *—(CH₂CH₂O)_{z}H group) are independently chosen from an integer chosen from 1, 2, 3, 4, 5, 6, 7, 8, 9, 10.

It is furthermore preferable according to the invention for the sum of x and y according to formula (X) (or if R′ is a *—(CH₂CH₂O)_{z}H group, the sum of x and y and z) to be a number from 3 to 20, particularly a number from 5 to 15.

A particularly preferred cationic surfactant has the formula (Xa)—

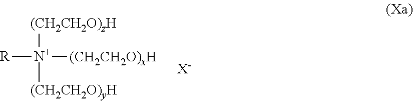

wherein
x, y and z are independently an integer number greater than 0 and the sum of (x+y+z) is a number from 3 to 20, particularly from 5 to 15,
R is a (C₈ to C₂₀) alkyl group or a (C₈ to C₂₀) alkenyl group, and
X⁻ is a physiologically tolerable anion.

A most particularly preferred cationic surfactant is the tris(oligooxyethyl)alkylammonium dihydrogen phosphate salt having the molecular weight of 780 g/mol, which under the INCI name Quaternium-52 is sold, for example, by Cognis under the trade name Dehyquart® SP in the form of an aqueous solution with 50 wt. % active substance.

A particularly preferred embodiment is therefore an agent which, in a cosmetically acceptable carrier, contains—
(a) at least one amphiphilic, cationic polymer having at least one structural unit of formula (I), at least one structural unit of formula (II-8), and at least one structural unit of formula (III-8)

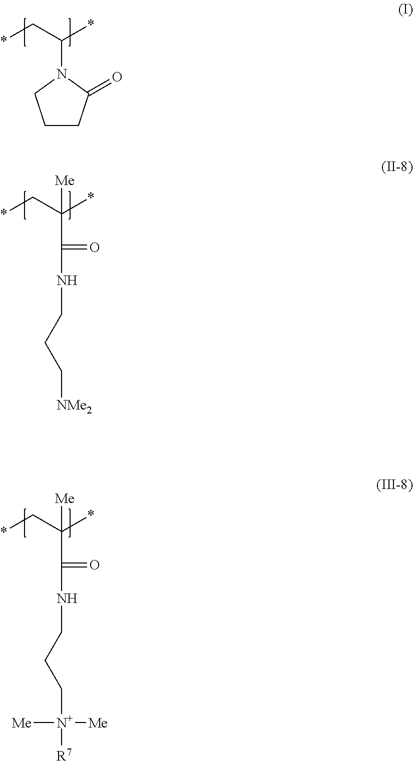

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl),
(b) at least one cationic polymer comprising at least one structural unit of formula (I), at least one structural unit of formula (V) and at least one structural unit of formula (VI),

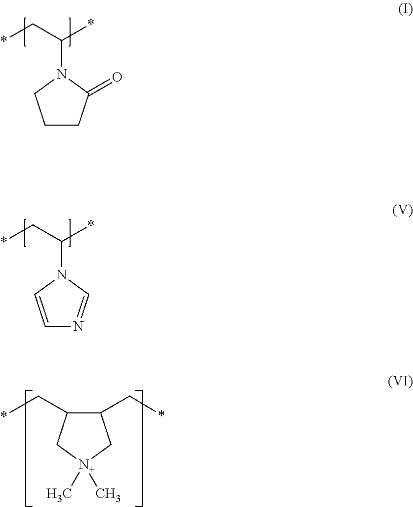

and
(c) at least one cationic surfactant.

Particularly preferred furthermore is an agent which, in a cosmetically acceptable carrier, contains—
(a) at least one amphiphilic, cationic polymer having at least one structural unit of formula (I), at least one structural unit of formula (II-8) and at least one structural unit of formula (III-8)

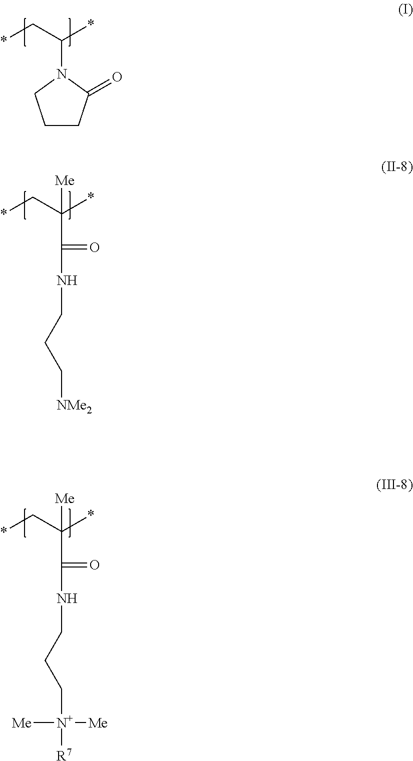

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl),
(b) at least one cationic polymer comprising at least one structural unit of formula (I), at least one structural unit of formula (V) and at least one structural unit of formula (VI),

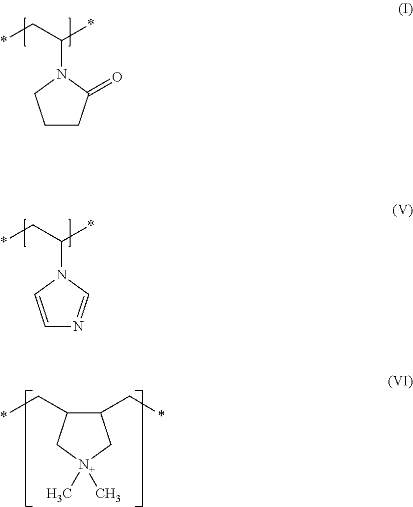

and
(c) at least one cationic surfactant according to formula (Xa)

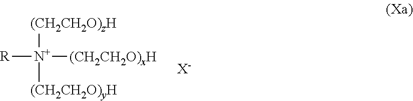

wherein
x, y and z are independently an integer number greater than 0 and the sum of (x+y+z) is a number from 3 to 20, particularly from 5 to 15,
R is a (C₈ to C₂₀) alkyl group or a (C₈ to C₂₀) alkenyl group, and
X⁻ is a physiologically tolerable anion.

Most particularly preferred is furthermore an agent which, in a cosmetically acceptable carrier, contains—
(a) at least one amphiphilic, cationic polymer having at least one structural unit of formula (I), at least one structural unit of formula (II-8) and at least one structural unit of formula (III-8)

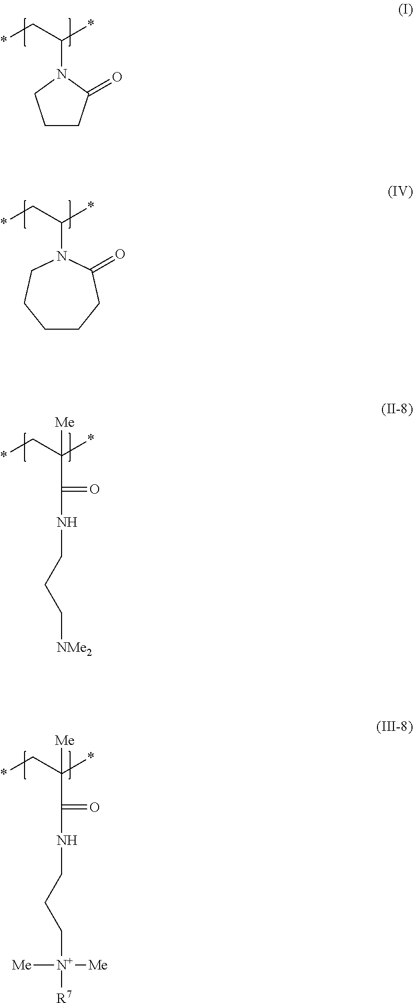

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl),
(b) at least one cationic polymer comprising at least one structural unit of formula (I), at least one structural unit of formula (V) and at least one structural unit of formula (VI),

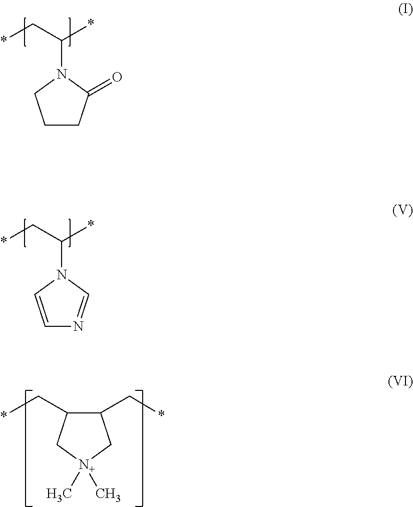

and
(c) at least one cationic surfactant according to formula (Xa)

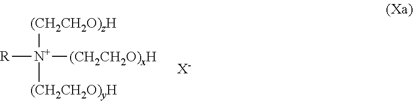

Surface-active compounds classified as zwitterionic surfactants have at least one quaternary ammonium group and at least one —COO⁽⁻⁾ or —SO₃⁽⁻⁾ group in the molecule. Particularly suitable zwitterionic surfactants are betaines such as N-alkyl-N,N-dimethylammonium glycinates, for example, cocoalkyl dimethylammonium glycinate, N-acyl aminopropyl-N,N-dimethylammonium glycinates, for example cocoacylaminopropyl dimethylammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name Cocamidopropyl Betaine.

Ampholytes are surface-active compounds which, in addition to a C₈-C₂₄ alkyl or acyl group, contain at least one free amino group and at least one —COOH or —SO₃H group in the molecule and are capable of forming internal salts. Examples of suitable ampholytes are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Particularly preferred ampholytes are N-cocoalkyl aminopropionate, cocoacylaminoethyl aminopropionate and C₁₂-C₁₈ acyl sarcosine.

Agents according to the invention contain the ingredients and active ingredients in a cosmetically acceptable carrier.

Preferred cosmetically acceptable carriers are aqueous, alcoholic or aqueous-alcoholic media having preferably at least 10 wt. % water, relative to the total agent. The low alcohols having 1 to 4 carbon atoms that are conventionally used for cosmetic purposes, such as ethanol and isopropanol, can be included in particular as alcohols.

It is preferable according to the invention to use at least one (C₁ to C₄) monoalkyl alcohol in the agents, particularly in an amount from 1 to 50 wt. %, more particularly from 5 to 30 wt. %. This is preferable for the formulation as a pump foam or aerosol foam.

Organic solvents or a mixture of solvents with a boiling point of 400° C. or lower can be included as additional co-solvents in an amount from 0.1 to 15 percent by weight, preferably from 1 to 10 percent by weight, based on total agent. Unbranched or branched hydrocarbons, such as pentane, hexane, isopentane, and cyclic hydrocarbons, such as cyclopentane and cyclohexane, are particularly suitable as additional co-solvents. Other particularly preferred water-soluble solvents are glycerol, ethylene glycol and propylene glycol in an amount of up to 30 wt. % relative to the total agent.

In particular, the addition of glycerol and/or propylene glycol and/or polyethylene glycol and/or polypropylene glycol increases the flexibility of the polymer film formed on application of the agent according to the invention. If a flexible hold is desired, the agents according to the invention therefore preferably contain 0.01 to 30 wt. % of glycol and/or propylene glycol and/or polyethylene glycol and/or polypropylene glycol, relative to the total agent.

The agents preferably have a pH of 2 to 11. The pH range from 2 to 8 is particularly preferred. Unless otherwise specified, within the meaning of this document the pH values stated relate to the pH at 25° C.

Agents according to the invention can also contain auxiliary substances and additives that are conventionally added to customary styling agents.

Suitable auxiliary substances and additives include in particular additional care substances.

Silicone oil and/or a silicone gum, for example, can be used as a care substance.

Suitable silicone oils or silicone gums according to the invention are in particular dialkyl and alkylaryl siloxanes, such as dimethyl polysiloxane and methyl phenyl polysiloxane, and the alkoxylated, quaternized or also anionic derivatives thereof. Cyclic and linear polydialkyl siloxanes, the alkoxylated and/or aminated derivatives thereof, dihydroxypolydimethyl siloxanes and polyphenyl alkyl siloxanes are preferred.

Silicone oils give rise to a wide variety of effects. Thus, for example, they influence simultaneously the dry and wet combability of hair, the feel of dry and wet hair and the gloss. The term “silicone oils” is understood by one skilled in the art to mean a plurality of structures of organosilicon compounds. They are firstly understood to be dimethiconols.

The following commercial products are cited as examples of such products: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzenesulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401 DC (all of the above Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all of the above Dow Corning Corporation), Dub Gel SI 1400 (Stearinerie Dubois Fils), HVM 4852 Emulsion (Crompton Corporation), Jeesilc 6056 (Jeen International Corporation), Lubrasil, Lubrasil DS (both Guardian Laboratories), Nonychosine E, Nonychosine V (both Exsymol), SanSurf Petrolatum-25, Satin Finish (both Collaborative Laboratories, Inc.), Silatex-D30 (Cosmetic Ingredient Resources), Silsoft 148, Silsoft E-50, Silsoft E-623 (all of the above Crompton Corporation), SM555, SM2725, SM2765, SM2785 (all of the above GE Silicones), Taylor T-Sil CD-1, Taylor TME-4050E (all Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all of the above Wacker-Chemie GmbH).

Dimethicones form the second group of silicones that can be included according to the invention. These can be both linear and branched and also cyclic or cyclic and branched.

Dimethicone copolyols (S3) are a further group of silicones that are suitable. Corresponding dimethicone copolyols are commercially available and are sold, for example, by Dow Corning under the name Dow Corning® 5330 Fluid.

The teaching according to the invention naturally also includes the fact that the dimethiconols, dimethicones and/or dimethicone copolymers can already be in the form of an emulsion. The corresponding emulsion of dimethiconols, dimethicones and/or dimethicone copolyols can be produced both after production of the corresponding dimethiconols, dimethicones and/or dimethicone copolyols and by the conventional emulsification methods known to one skilled in the art. To this end, cationic, anionic, non-ionic or zwitterionic surfactants and emulsifiers as auxiliary substances can be used as auxiliary agents to produce the corresponding emulsions. Emulsions of the dimethiconols, dimethicones and/or dimethicone copolyols can also be produced directly by an emulsion polymerization method. Such methods are known to one skilled in the art.

If the dimethiconols, dimethicones and/or dimethicone copolyols are used as an emulsion, then according to the invention the droplet size of the emulsified particles is 0.01 to 10,000 μm, preferably 0.01 to 100 μm, more preferably 0.01 to 20 μm and even more preferably 0.01 to 10 μm. Particle size is determined by the light scattering method.

If branched dimethiconols, dimethicones and/or dimethicone copolyols are used, this should be understood to mean that the branching is greater than a random branching that occurs by chance due to impurities in the various monomers. Within the meaning of the present invention branched dimethiconols, dimethicones and/or dimethicone copolyols are therefore understood to have a degree of branching greater than 0.01%. A degree of branching greater than 0.1% is preferred, most particularly preferably greater than 0.5%. The degree of branching is determined from the ratio of unbranched monomers to branched monomers, in other words the amount of trifunctional and tetrafunctional siloxanes. Both low-branched and highly branched dimethiconols, dimethicones and/or dimethicone copolyols can be most particularly preferred according to the invention.

Particularly suitable silicones are amino-functional silicones, in particular the silicones that are grouped together under the INCI name amodimethicones. It is therefore preferable according to the invention for the agents according to the invention additionally to contain at least one amino-functional silicone. These are understood to be silicones having at least one, optionally substituted, amino group. Under the INCI declaration these silicones are known as amodimethicones and they are available for example in the form of an emulsion as the commercial product Dow Corning® 939 or as the commercial product Dow Corning® 949 mixed with a cationic and a non-ionic surfactant.

Amino-functional silicones are preferably used that have an amine value above 0.25 meq/g, preferably above 0.3 meq/g and in particular preferably above 0.4 meq/g. The amine value denotes the milli-equivalents of amine per gram of the amino-functional silicone. It can be determined by titration and also specified in the unit mg KOH/g.

Agents contain the silicones preferably in amounts from 0.01 wt. % to 15 wt. %, particularly preferably from 0.05 to 2 wt. %, based on total agent.

As a care substance from a different class of compounds, the agent can, for example, contain at least one protein hydrolysate and/or a derivative thereof.

Protein hydrolysates are mixtures of products obtained by acidically, basically or enzymatically catalyzed breakdown of proteins. According to the invention, the term protein hydrolysates also includes total hydrolysates and individual amino acids and derivatives thereof as well as mixtures of different amino acids. The molecular weight of protein hydrolysates for use according to the invention is from 75, the molecular weight for glycine, and 200,000; the molecular weight is preferably 75 to 50,000 and more preferably 75 to 20,000 Daltons.

According to the invention, protein hydrolysates of both plant and animal or marine or synthetic origin can be used.

Animal protein hydrolysates include elastin, collagen, keratin, silk and milk protein hydrolysates, which can also be present in the form of salts. Such products are sold, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inalex), Sericin (Pentapharm) and Kerasol® (Croda).

The protein hydrolysates are present in agents according to the invention in amounts, for example, from 0.01 wt. % to 20 wt. %, preferably from 0.05 wt. % to 15 wt. % and more preferably from 0.05 wt. % to 5 wt. %, based on total application preparation.

Agents according to the invention can furthermore contain at least one vitamin, provitamin, vitamin precursor and/or derivative thereof as a care substance.

Such vitamins, provitamins and vitamin precursors are preferred according to the invention that are conventionally assigned to groups A, B, C, E, F and H.

The agents according to the invention preferably contain vitamins, provitamins and vitamin precursors from groups A, B, C, E and H. Panthenol, pantolactone, pyridoxine and derivatives thereof as well as nicotinic acid amide and biotin are particularly preferred.

D-Panthenol, optionally in combination with at least one of the aforementioned silicone derivatives, is most particularly preferably used as a care substance.

Like the addition of glycerol and/or propylene glycol, the addition of panthenol increases the flexibility of the polymer film formed on application of the agent according to the invention. If a particularly flexible hold is desired, the agents according to the invention can therefore contain panthenol instead of or in addition to glycerol and/or propylene glycol. In a preferred embodiment the agents according to the invention contain panthenol, preferably in an amount from 0.05 to 10 wt. %, particularly preferably 0.1 to 5 wt. %, relative in each case to the total agent.

Agents according to the invention can moreover contain at least one plant extract as a care substance.

These extracts are conventionally produced by extraction of the entire plant. It can also be preferable in individual cases, however, to produce the extracts exclusively from flowers and/or leaves of the plant.

Extracts from green tea, oak bark, stinging nettle, witch hazel, hops, henna, chamomile, burdock, horsetail, whitethorn, lime blossom, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root are preferred above all according to the invention.

It can furthermore be preferred to use mixtures of a plurality of different plant extracts, particularly two, in agents according to the invention.

Mono- or oligosaccharides can also be used as a care substance in the agents according to the invention.

Both monosaccharides and oligosaccharides, such as cane sugar, lactose and raffinose, can be used. Use of monosaccharides is preferred according to the invention. Of the monosaccharides, compounds containing 5 or 6 carbon atoms are preferred in turn.

Suitable pentoses and hexoses include ribose, arabinose, xylose, lyxose, allose, altrose, glucose, mannose, gulose, idose, galactose, talose, fucose and fructose. Arabinose, glucose, galactose and fructose are preferably used carbohydrates. Glucose, which is suitable both in the D-(+)- or L-(−)- configuration or as a racemate, is most particularly preferably used.

Derivatives of these pentoses and hexoses, such as the corresponding aldonic and uronic acids (sugar acids), sugar alcohols and glycosides, can moreover also be used according to the invention. Preferred sugar acids are gluconic acid, glucuronic acid, saccharic acid, mannosaccharic acid and mucic acid. Preferred sugar alcohols are sorbitol, mannitol and dulcitol. Preferred glycosides are methyl glucosides.

As the mono- or oligosaccharides that are used are conventionally obtained from natural raw materials such as starch, they generally have the configurations corresponding to these raw materials (for example D-glucose, D-fructose and D-galactose).

The mono- or oligosaccharides are preferably present in agents according to the invention in an amount from 0.1 to 8 wt. %, particularly 1 to 5 wt. %, based on total application preparation.

The agent can moreover contain at least one lipid as a care substance.

Lipids that are suitable according to the invention are phospholipids, for example, soy lecithin, egg lecithin and cephalins, and the substances known under the INCI names Linoleamidopropyl PG-Dimonium Chloride Phosphate, Cocamidopropyl PG-Dimonium Chloride Phosphate and Stearamidopropyl PG-Dimonium Chloride Phosphate. These are sold, for example, by Mona under the commercial names Phospholipid EFA®, Phospholipid PTC® and Phospholipid SV®. Agents according to the invention contain the lipids preferably in amounts from 0.01 to 10 wt. %, particularly 0.1 to 5 wt. %, relative to the total application preparation.

Oil bodies are also suitable as a care substance.

Natural and synthetic cosmetic oil bodies include:
vegetable oils. Examples of such oils are sunflower oil, olive oil, soybean oil, rapeseed oil, almond oil, jojoba oil, orange oil, wheat germ oil, peach kernel oil and the liquid components of coconut butter. Other triglyceride oils are also suitable, however, such as the liquid components of beef fat and synthetic triglyceride oils.
liquid paraffin oils, isoparaffin oils and synthetic hydrocarbons and also di-n-alkyl ethers having in total between 12 and 36 C atoms, in particular between 12 and 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and also di-tert-butyl ether, diisopentyl ether, di-3-ethyl decyl ether, tert-butyl-n-octyl ether, isopentyl-n-octyl ether and 2-methyl pentyl-n-octyl ether. The compounds available as commercial products, 1,3-di-(2-ethylhexyl)cyclohexane (Cetiol® S) and di-n-octyl ether (Cetiol® OE), can be preferred.
ester oils. Ester oils are understood to be the esters of C₆-C₃₀ fatty acids with C₂-C₃₀ fatty alcohols. The monoesters of fatty acids with alcohols having 2 to 24 C atoms are preferred. Particularly preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).
dicarboxylic acid esters such as di-n-butyl adipate, di-(2-ethylhexyl) adipate, di-(2-ethylhexyl) succinate and diisotridecyl acelate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate;
symmetrical, asymmetrical or cyclic esters of carbonic acid with fatty alcohols, as described, for example, in DE-OS 197 56 454, glycerol carbonate or dicaprylyl carbonate (Cetiol® CC);
tri-fatty acid esters of saturated and/or unsaturated linear and/or branched fatty acids with glycerol;
fatty acid partial glycerides, namely monoglycerides, diglycerides and technical mixtures thereof. If technical products are used, small amounts of triglycerides may also be included for production reasons. The partial glycerides are preferably of the formula (D4-I),

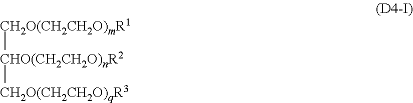

wherein R¹, R² and R³ are independently hydrogen or a linear or branched, saturated and/or unsaturated acyl residue having 6 to 22, preferably 12 to 18, carbon atoms, with the proviso that at least one of these groups is an acyl residue and at least one of these groups is hydrogen. The sum (m+n+q) is 0 or a number from 1 to 100, preferably 0 or 5 to 25. R¹ is preferably an acyl residue and R² and R³ hydrogen, and the sum (m+n+q) is preferably 0. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Oleic acid monoglycerides are preferably used.

The amount of natural and synthetic cosmetic oil bodies used in agents according to the invention is typically 0.1 to 30 wt. %, preferably 0.1 to 20 wt. % and in particular 0.1 to 15 wt. %, based on total application preparation.

Although each of the specified care substances in itself already gives rise to a satisfactory result, all embodiments in which the agent contains a plurality of care substances, including examples from different groups, are also included in the context of the present invention.

With the addition of a UV filter, both the agents and the treated fibers can be protected from damaging influences of UV radiation. At least one UV filter is therefore preferably added to the agent. There are no general restrictions on the suitable UV filters in terms of their structure and their physical properties. In fact all UV filters that can be used in the cosmetics sector whose absorption maximum is in the UVA (315-400 nm), UVB (280-315 nm) or UVC (<280 nm) range are suitable. UV filters having an absorption maximum in the UVB range, in particular in the range from approximately 280 to approximately 300 nm, are particularly preferred.

Preferred UV filters according to the invention include substituted benzophenones, p-aminobenzoic acid esters, diphenyl acrylic acid esters, cinnamic acid esters, salicylic acid esters, benzimidazoles and o-aminobenzoic acid esters.

The UV filters are typically included in amounts from 0.01 to 5 wt. %, based on total application preparation. Amounts from 0.1 to 2.5 wt. % are preferred.

In a preferred embodiment the agent according to the invention also contains one or more substantive dyes. This allows the treated keratinic fibers not only to be temporarily structured but at the same time also to be colored through the use of the agent. This can be desirable in particular if only a temporary coloration is desired, for example with striking fashion colors, which can be removed again from the keratinic fibers simply by washing.

Substantive dyes are conventionally nitrophenylene diamines, nitroaminophenols, azo dyes, anthraquinones or indophenols. Preferred substantive dyes are the compounds known under the international names or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 11, HC Blue 12, Disperse Blue 3, Acid Blue 7, Acid Green 50, HC Violet 1, Disperse Violet 1, Disperse Violet 4, Acid Violet 43, Disperse Black 9, Acid Black 1 and Acid Black 52 as well as 1,4-diamino-2-nitrobenzene, 2-amino-4-nitrophenol, 1,4-bis-(1-hydroxyethyl)-amino-2-nitrobenzene, 3-nitro-4-(1′-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)amino-5-chloro-2-nitrobenzene, 4-amino-3-nitrophenol, 1-(2′-ureidoethyl)amino-4-nitrobenzene, 4-amino-2-nitro-diphenylamine-2′-carboxylic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid and 2-chloro-6-ethylamino-1-hydroxy-4-nitrobenzene. Cationic substantive dyes are preferably used. Of those, the following are particularly preferred:
(a) cationic triphenylmethane dyes, such as Basic Blue 7, Basic Blue 26, Basic Violet 2 and Basic Violet 14;
(b) aromatic systems substituted with a quaternary nitrogen group, such as Basic Yellow 57, Basic Red 76, Basic Blue 99, Basic Brown 16 and Basic Brown 17; and
(c) substantive dyes containing a heterocyclic compound having at least one quaternary nitrogen atom, such as those mentioned in EP-A2-998 908, particularly in claims 6 to 11 therein.

Dyes also known under the names Basic Yellow 87, Basic Orange 31 and Basic Red 51 are most particularly preferred cationic substantive dyes of group (c). Cationic substantive dyes which are sold under the trademark Arianor® are likewise most particularly preferred cationic substantive dyes according to the invention.

Agents according to this embodiment preferably contain substantive dyes in an amount from 0.001 to 20 wt. %, based on total agent.

Preferably, agents according to the invention are free from oxidation dye precursors. Oxidation dye precursors are divided into developer components and coupler components. The developer components form the actual dyes under the influence of oxidizing agents or of atmospheric oxygen with one another or by coupling with one or more coupler components.

Agents according to the invention can be formulated in all conventional forms for styling agents, for example, as solutions which can be applied to the hair as a hair lotion or pump or aerosol spray, as creams, emulsions, waxes, gels or surfactant-containing foaming solutions, or as other preparations suitable for application on the hair.

Hair creams and hair gels generally contain structuring agents and/or thickening polymers, which serve to impart the desired consistency to the products. Structuring agents and/or thickening polymers are typically used in an amount from 0.1 to 10 wt. %, based on total product. Amounts from 0.5 to 5 wt. %, particularly 0.5 to 3 wt. %, are preferred.

Agents according to the invention are preferably formulated as a pump spray, aerosol spray, pump foam or aerosol foam.

To this end agents according to the invention are presented in a dispensing device, which is either a compressed gas cylinder (aerosol container) additionally filled with a propellant or a non-aerosol container.

Compressed gas cylinders, by which a product is distributed through a valve via the internal gas pressure in the cylinder, are termed “aerosol containers”. In contrast to the aerosol definition, a “non-aerosol container” is a container under normal pressure, by which a product is distributed through a pump system by mechanical action.

Agents according to the invention are preferably formulated as an aerosol hair foam or aerosol hair spray. The agent therefore preferably additionally contains at least one propellant.

Suitable propellants according to the invention include N₂O, dimethyl ether, CO₂, air, alkanes having 3 to 5 carbon atoms, such as propane, n-butane, isobutane, n-pentane and isopentane, and mixtures thereof. Dimethyl ether, propane, n-butane, isobutane and mixtures thereof are preferred.

According to a preferred embodiment the specified alkanes, mixtures of the specified alkanes or mixtures of the specified alkanes with dimethyl ether are used as the sole propellant. However, the invention expressly also includes the incorporation of chlorofluorocarbon propellants, particularly fluorocarbons.

In the given spray device, the sizes of the aerosol droplets or foam bubbles and the size distribution can be adjusted by the ratio of propellant to the other ingredients of the preparations.

The amount of propellant used varies according to the specific composition of the agent, the packaging used and the desired product type (e.g., hair spray or hair foam). If conventional spray devices are used, aerosol foam products preferably contain propellant in amounts from 1 to 35 wt. %, based on total product. Amounts from 2 to 30 wt. %, particularly 3 to 15 wt. %, are particularly preferred. Aerosol sprays generally contain larger amounts of propellant. In this case the propellant is preferably used in an amount from 30 to 98 wt. %, based on total product. Amounts from 40 to 95 wt. %, particularly 50 to 95 wt. %, are particularly preferred.

The aerosol products can be produced using conventional techniques. All ingredients of the individual agent with the exception of the propellant are generally introduced into a suitable pressure-resistant container. This is then closed with a valve. Finally, the desired amount of propellant is added using conventional techniques.

Isopentane is preferably suitable as a propellant for expanding agents in gel form in a two-chamber aerosol container, it being incorporated into the agents according to the invention and introduced into the first chamber of the two-chamber aerosol container. At least one further propellant different from isopentane and that provides a higher pressure in the two-chamber aerosol container than isopentane is introduced into the second chamber of the two-chamber aerosol container. The propellants of the second chamber are preferably chosen from N₂O, dimethyl ether, CO₂, air, alkanes having 3 or 4 carbon atoms (such as propane, n-butane, isobutane) and mixtures thereof.

Aerosol hair foams or aerosol hair sprays containing the agents according to the invention as described above and at least one propellant are a preferred embodiment of the agents according to the invention.

Preferred agents according to the invention and propellants of the aerosol hair foam or aerosol hair spray and the amounts of propellant correspond to the details specified above.

The invention secondly provides for use of the agents according to the invention for temporarily shaping hair and/or for hair care.

Agents according to the invention and products containing these agents, particularly aerosol hair foams or aerosol hair sprays, impart a very strong, lasting styling hold to the treated hair, even though the hair remains flexible. If the agent is formulated as hair foam, a stable, fine-pored and creamy foam is formed, which can be distributed evenly onto the hair without dripping.

The invention thirdly provides a method for treating keratin-containing fibers, particularly human hair, wherein, using a dispensing device, an agent according to the first subject-matter of the invention is expanded into foam and the resulting foam is applied to the keratin-containing fibers.

Preferably, the keratin-containing fibers are shaped and this shape is fixed by the agent of the first subject-matter of the invention.

The aforementioned dispensing devices (see above) are preferred according to the invention.

The invention fourthly provides a method for treating keratin-containing fibers, particularly human hair, wherein, using a dispensing device, an agent according to the first subject-matter of the invention is applied to the keratin-containing fibers as a spray.

Preferably, the keratin-containing fibers are shaped and this shape is fixed by the agent of the first subject-matter of the invention.

The aforementioned dispensing devices (see above) are preferred according to the invention.

The examples below are intended to illustrate the subject-matter of the present invention without in any way limiting it.

### Examples

Unless otherwise specified, the quantities given below are percentages by weight.

The following formulations were prepared:

| Raw materials | E1 | E2 | E3 | E4 |
|---|---|---|---|---|
| Styleze ® W10 | 5.0 | 5.0 | — | 5.0 |
| AquaStyle ® 300 | 5.0 | — | 5.0 | 5.0 |
| Luviquat ® Sensation | 0.3 | 0.3 | 0.3 | 0.3 |
| Dehyquart ® SP | — | — | — | 0.5 |
| Perfume | 0.2 | 0.2 | 0.2 | 0.2 |
| Propane/butane¹ | 6.0 | 6.0 | 6.0 | 6.0 |
| Water | 83.5 | 88.5 | 88.5 | 83.0 |

¹Propane/butane mixture (47 wt. % propane, 50 wt. % butane, 3 wt. % isobutane)

Regarding the composition of the raw materials used, reference is made to the details specified above.

The formulation ingredients for each formulation were introduced into separate aerosol containers that satisfy the following technical parameters: aluminum storage vessel with valve, product 522983 PV10697 from Precision (Deutsche Präzisions-Ventil GmbH).

The formulations were each dispensed from the aerosol containers as hair foam and distributed on the hair of a test subject. The formulations were perceived to be gentle on the skin and when applied to the hair brought about a styling hold. A very good care effect on the hair was obtained.

We claim:

1. An agent for treating keratin-containing fibers comprising in a cosmetically acceptable carrier:
(a) at least one amphiphilic, cationic polymer having at least one structural unit of formula (I), at least one structural unit of formula (II) and at least one structural unit of formula (III), 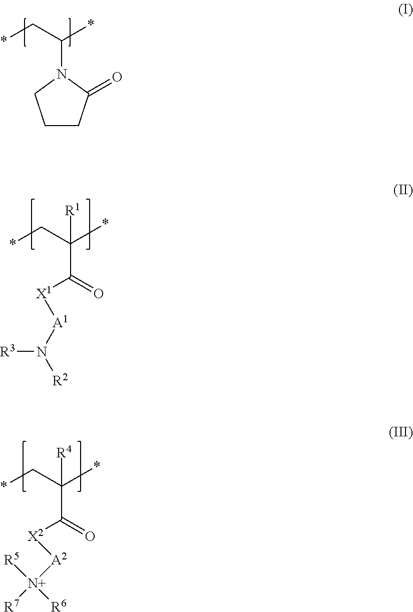
wherein
R¹ and R⁴ are independently a hydrogen atom or a methyl group,
X¹ and X² are independently an oxygen atom or an NH group,
A¹ and A² are independently an ethane-1,2-diyl, propane-1,3-diyl or butane-1,4-diyl group,
R², R³, R⁵ and R⁶ are independently a (C₁ to C₄) alkyl group, and
R⁷ is a (C₈ to C₃₀) alkyl group
and
(b) at least one cationic polymer having at least one structural unit of formula (I), at least one structural unit of formula (V) and at least one structural unit of formula (VI),

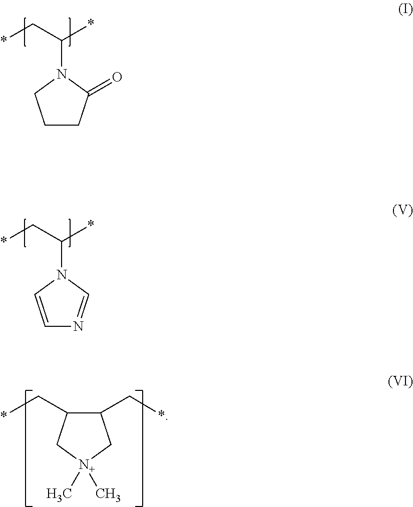

2. The agent according to claim 1, wherein R¹ and R⁴ are each a methyl group.

3. The agent according to claim 1, wherein A¹ and A² are independently ethane-1,2-diyl or propane-1,3-diyl.

4. The agent according to claim 1, wherein X¹ is an NH group.

5. The agent according claim 1, wherein X² is an NH group.

6. The agent according to claim 1, wherein R², R³, R⁵ and R⁶ are independently methyl or ethyl.

7. The agent according to claim 1, wherein the amphiphilic, cationic polymer comprises at least one structural unit of formula (I), at least one structural unit of formula (II-8) and at least one structural unit of formula (III-8) 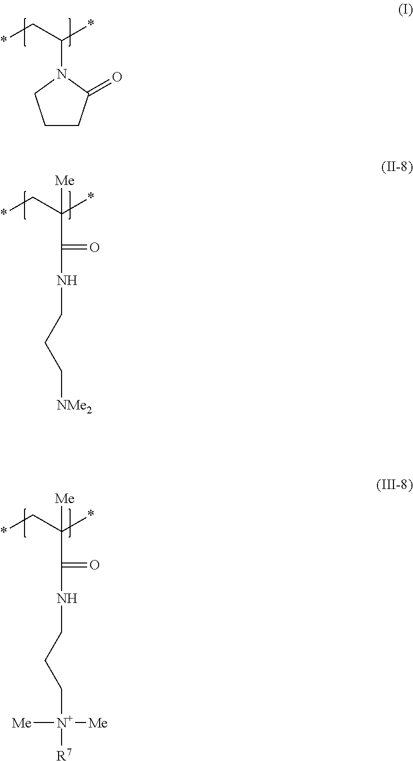
wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl).

8. The agent according to claim 1, wherein the amphiphilic, cationic polymer further comprises at least one structural unit of formula (IV)

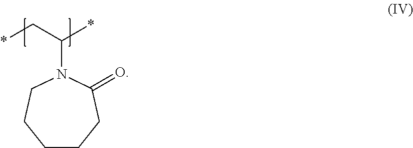

9. The agent according to claim 1, wherein the amphiphilic, cationic polymers are present in an amount from 0.1 wt. % to 20.0 wt. %, based on total weight of the agent.

10. The agent according to claim 1, wherein the cationic polymer is at least a cationic polymer having the INCI name Polyquaternium-87.

11. The agent according to claim 1, wherein the cationic polymers are present in an amount from 0.01 wt. % to 20 wt. %, based on total weight of the agent.

12. An aerosol foam or aerosol spray comprising the agent according to claim 1.

13. A method for treating keratin-containing fibers comprising expanding the agent according to claim 1 into a foam using a dispensing device, and applying the resulting foam to the keratin-containing fibers.

14. A method for treating keratin-containing fibers comprising applying the agent according to claim 1 as a spray using a dispensing device to the keratin-containing fibers.
